(19) United States
(12) Patent Application Publication
(10) Pub. No.: US 2023/0197222 A1
(43) Pub. Date: Jun. 22, 2023

(54) SYSTEMS AND METHODS FOR CHANGING A PRESCRIBED MEDICATION FOR A PATIENT

(71) Applicant: Gemini Health LLC, Sausalito, CA (US)

(72) Inventors: Edward FOTSCH, Sausalito, CA (US); Roger PINSONNEAULT, Johns Creek, GA (US); Dwight DAVIS, Little Rock, AR (US)

(21) Appl. No.: 18/168,469

(22) Filed: Feb. 13, 2023

Related U.S. Application Data
(63) Continuation-in-part of application No. 17/402,406, filed on Aug. 13, 2021, now Pat. No. 11,581,076.
Application No. 17/402,406 is a continuation of application No. 16/036,851, filed on Jul. 16, 2018, now Pat. No. 11,127,490.
(60) Provisional application No. 63/420,137, filed on Oct. 28, 2022.
(60) Provisional application No. 62/618,296, filed on Jan. 17, 2018.
(60) Provisional application No. 62/645,350, filed on Mar. 20, 2018.
(60) Provisional application No. 62/667,141, filed on May 4, 2018.
(60) Provisional application No. 62/670,146, filed on May 11, 2018.

(51) Int. Cl. G16H 10/60; G16H 20/10

(52) U.S. Cl. CPC G16H 10/60; G16H 20/10

(57) ABSTRACT

Methods and apparatuses are disclosed for changing a prescribed medication for a patient. These methods and apparatuses may interface with a patient's electronic health records and payer databases and identify alternative therapeutically equivalent medications to replace a patient's existing medications with less expensive alternatives. The patient's care team can interactively manipulate and modify the data to determine alternatives.

### CLAIM OF PRIORITY

This patent application claims priority to U.S. Provisional Patent Application No. 63/420,137, filed on Oct. 28, 2022 (titled “SYSTEMS AND METHODS FOR DYNAMIC INTERACTIVE DRUG SAVINGS REPORTS SERVICES”). This patent application also claims priority as a continuation-in-part to U.S. patent application Ser. No. 17/402,406, now U.S. Pat. No. 11,581,076, titled “METHODS AND APPARATUSES FOR PROVIDING ALTERNATIVES FOR PREEXISTING PRESCRIBED MEDICATIONS,” filed on Aug. 13, 2021, which claimed priority as a continuation of U.S. patent application Ser. No. 16/036,851, now U.S. Pat. No. 11,127,490, titled “METHODS AND APPARATUSES FOR PROVIDING ALTERNATIVES FOR PREEXISTING PRESCRIBED MEDICATIONS,” filed Jul. 16, 2018, which claims priority to U.S. Provisional Patent Application No. 62/618,296, filed on Jan. 17, 2018, titled “AUTOMATED ALTERNATIVES ANALYSIS,” and U.S. Provisional Patent Application No. 62/645,350, filed Mar. 20, 2018, titled “SYSTEMS AND METHODS FOR AUTOMATED ALTERNATIVES IDENTIFICATION FOR EXISTING PRESCRIPTION DRUGS AND RELATED COSTS.” U.S. patent application Ser. No. 16/036,851 also claims priority to U.S. Provisional Patent Application No. 62/667,141, filed on May 4, 2018 (titled “SYSTEMS AND METHODS FOR CONFIGURABLE PRESCRIPTION ALTERNATIVE IDENTIFICATION AND MANAGEMENT/PROVIDER AND PAYER CONFIGURATION ALTERNATIVE DIAGNOSIS USE OF CHOSEN MEDICATIONS”), and U.S. Provisional Patent Application No. 62/670,146, filed May 11, 2018 (titled “AUTOMATED ALTERNATIVES ANALYSIS (AAA) AND DRUG SAVINGS REPORT (DSR)”). Each of these patents and patent application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The methods and apparatuses described herein generally relate to automated modification of existing patient medications and pricing transparency, and more specifically to methods and apparatuses to identify and prioritize alternatives for prescribed medications.

### BACKGROUND

Health costs continue to grow in the U.S. and are well documented as a formidable national challenge, with prescription drug costs among the leading sources of cost escalation. Currently, Prescribers are typically notified to renew existing prescriptions for their patients by phone call, fax or electronic systems when an existing prescription has expired, but cost information is generally not provided to the Prescriber, even if the cost of the medication has gone up dramatically and/or lower cost therapeutically equivalent medications are available. Alternative drug or fulfillment cost information is not routinely or automatically delivered to Prescribers or patients related to prescribing new medications or for evaluating the cost-effectiveness of existing medications. Prescribers cannot consider cost-efficiency when prescribing or renewing prescription, or at the point of care. Drug costs and cost savings opportunities are not currently considered for new or existing prescriptions, and as a result drug costs continue to escalate.

For most drug categories there are numerous drug alternatives that a Prescriber might consider in treating a patient. Many Prescribers are not completely familiar with all drugs in every category in which they prescribe. And since Prescribers are given neither a list of alternatives to consider, nor the cost of alternative drugs, both clinical and economic efficiency are lacking.

Drug costs can vary by many factors including: drug chosen, form of drug (long acting, etc.), patient's insurance, patient's deductible, including high deductible plans and Affordable Care Act (ACA) health plans, pharmacy and patient financial assistance programs. Drug costs are dynamic and change regularly, even between new scripts and renewal scripts. Drug costs may also vary depending on the fulfillment means used: e.g., $4 generics (Wal-Mart, Target, etc.), pharmacy discount card or mail order, etc. The pricing of a medication can be complicated.

The optimal alternative drug for a given patient may vary substantially based upon payer (e.g., insurer, health organizations, etc.) formularies, payer preferences and provider preferences, or because new drugs are approved, or new uses of existing medications are found or approved.

A prescription drug and the pharmacy used to fill the prescription may be selected via an e-Prescribing or electronic health record (“EHR”) application. For example, a Prescriber may select the drug to be prescribed. The preferred pharmacy (e.g., a ‘pharmacy of last fill’) may be a default field in electronic prescribing workflow. The patient's preferred pharmacy is typically entered into the provider's EHR or prescribing application when the patient initially registers with the provider for medical care. The prescription may be sent electronically to the patient's chosen pharmacy.

While Prescribers have shown an interest in knowing drug costs, alternative drugs and their costs, and related clinical messages about the prescriptions to be created or renewed, Prescribers typically do not want to spend time considering all possible therapeutic alternatives and then price-shopping for each drug for each patient. Prescribers often have more than one drug in a specific clinical category that they use to treat a specific illness; such as using any of a number of different Statins to treat high cholesterol. Prescribers in these instances could select drugs that are more cost effective for the patient when a prescription is created or renewed if alternative medications and costs were provided.

There is a pressing need for identification and selection of therapeutic drug alternatives for existing medications, ideally at the time and point of care. In addition to addressing other issues in health care, the methods and apparatuses described herein may address this need.

Health costs continue to grow in the U.S. and are well documented as a formidable national challenge, with prescription drug costs among the leading sources of cost escalation. For patients taking prescription drugs, these costs can be materially addressed by interactively identifying lower cost therapeutically equivalent alternative drug options in the context of a strategy that is sometimes referred to as “cost transparency” for patients and their caregivers. Patients, however, are unable to change their prescriptions without a licensed prescriber. Prescribers, typically doctors, may have limited time and information available to enable them to select lower cost medications, and may have limited experience with some medications that may be lower cost for patients. Pharmacists, population health clinicians or other providers (collectively referred to herein as simply “pharmacists”) may have the scope of knowledge, expertise and responsibility to assist in identifying lower cost drugs for patients, however, they lack robust interactive tools to request, receive, customize, prioritize and act upon lower cost drug alternatives.

Currently, there is very limited alternative drug and alternate fulfillment cost information that is available on a routine or automated basis related to the cost-effectiveness of existing medications. Prescribers are typically notified to renew existing prescriptions for their patients by phone call, fax or electronic systems when the existing prescription has expired—but cost information or equivalent alternative drugs of lower cost are not typically provided to the prescriber, even if the cost of the medication—to the payer, employer or health plan and the patient—have gone up dramatically. In addition, prescribers often delegate renewals to clinical office staff who do not have the ability to change medications to lower cost alternatives even if presented during the renewal process. The prescriber's primary job is to focus on patient care, not drug costs.

Pharmacists who work with prescribers are often tasked with identifying and recommending lower cost alternative drugs for patients, but have no related interactive tools to proactively identify alternative drugs and identify savings on a patient-specific basis. These pharmacists may receive flat files that include spread sheets that a patient's current medications. Separately, the pharmacists may have static lists of formularies relevant to groups of patients. As for selecting alternatives to existing medications, pharmacists lack an interactive system that identifies one or more potential alternatives and can map daily doses and day's supply. Patient-specific drug costs and cost savings opportunities are not currently part of pharmacist's workflow for existing prescriptions. For these reasons, while pharmacists attempt to reduce the costs for existing medications, their ability is limited by the lack of a robust interactive tools, and as a result, drug costs continue to escalate.

For most drug categories, there are numerous drug alternatives that a prescriber might consider in treating a patient, although a prescriber may not have experience with all alternatives. Also, many drugs are used for more than one diagnosis, making the selection of alternatives more challenging since pharmacists and payers may not know the diagnosis for each patient. Pharmacists are familiar with all drugs and alternatives but lack patient-specific costs that include the patient's pharmacy benefits coverage, formularies, deductibles and copays. Pharmacists are therefore unable to give prescribers a list of alternatives to consider, nor the cost of alternative drugs, so both clinical and economic efficiency are lacking. Even if pharmacists are presented alternatives, they are unable to customize these alternatives to include drugs which they prefer or exclude alternatives in general, on behalf of a prescriber and/or on a patient-specific basis. Also, they lack the ability to change potential alternative drugs based upon the history or diagnosis being treated for each patient.

Drug costs can vary by many factors including: drug chosen, form of drug (tablet vs capsule, cream vs ointment, extended release, etc.), patient's insurance, patient's deductible-including high deductible plans and Affordable Care Act (ACA) health plans, pharmacy or pharmacy type (e.g. retail vs. mail) and patient financial assistance programs. Drug costs are dynamic and change all the time—even between new prescription renewal prescription orders. In addition, insured patients may find that their drug costs are lowered by using alternative means for fulfillment. For example, generics (Wal-Mart, Target, etc.), pharmacy discount card or mail order, etc. The determination of the pricing of a medication can be complicated.

The optimal alternative drug, at the optimal cost, for a given patient may vary substantially based upon payer formularies, payer preferences, pharmacists' preferences, prescriber preferences, patient clinical status and also medications previously taken by the patient.

In current pharmacist workflows, the pharmacist working for a medical group or payer, is tasked with lowering the drug costs for a population of patients under the care of prescribers. The provider group may have entered into an Accountable Care Organization (“ACO”) contract with a payer or other type of contract that puts the provider group at risk for the overall pharmacy spend or otherwise rewards provider groups for lowering drug cost. Yet, aside from simple payer pharmacy claims, pharmacists lack the interactive tools to proactively identify and customize lower cost alternatives to existing medications that are patient and payer specific including diagnosis and patient-specific drug costs. Absent these tools, some pharmacists struggle to effectively address rising drug costs for the patients.

While prescribers have shown an interest in knowing drug costs, alternative drugs, and related clinical messages about the prescriptions to be created or renewed (e.g., Prior Authorization Required, alternative drugs to consider, etc.), prescribers may not want to spend time considering all possible therapeutic alternatives and then price-shopping for each patient. Prescribers look to pharmacists to assist in identifying lower cost alternatives that are optimized for each patient.

Therefore, there is a pressing need for an interactive system and method for the identification and selection of therapeutic drug alternatives for a patient's existing medications. The system and method should consider costs, as well as payer formularies, payer preferences, pharmacists preferences, prescriber preferences, including preferred means and times of engagement, patient clinical status including diagnosis if available, and also medications previously taken by the patient. Furthermore, the system and method should also deliver the alternative drug information to pharmacists in a workflow, enabling the pharmacists to engage prescribers with recommended alternative drugs and fulfillment. There is a related need to provide simple interactive communications related to lower cost medication alternatives in a manner that considers prescriber preferred medications, and reduces to the extent possible, the prescriber's administrative tasks of contacting pharmacies and patients to implement a switch to lower cost, therapeutically equivalent medication. There is an additional need to provide information back to pharmacists confirming that existing drugs were switched to lower cost alternatives by identifying the alternatives in the payer's claims file.

### SUMMARY OF THE DISCLOSURE

The present invention relates to methods and apparatuses (e.g., devices, systems, etc., including software, firmware and/or hardware) that may help reduce health care costs, and particularly medication costs without compromising, and often actually improving, patient care by making care more affordable. For patients taking prescription drugs, these costs can be materially addressed by automatically identifying lower cost therapeutic equivalent alternative drug options for patients, prescribers (including providers or physicians), and or pharmacists.

The methods and apparatuses described herein may enable a patient a prescriber, or a pharmacist to manipulate and/or generate a drug savings report (“DSR”) which may identify therapeutic alternatives for existing prescription medications. The DSR may include costs as well as potential savings that may be associated with alternative medications.

These methods and apparatuses may increase cost-effective prescribing and thereby lower drug costs. These methods and apparatuses may provide prescribers, pharmacists, and patients more cost-effective, therapeutically equivalent alternatives for existing medications through one or more DSRs. Specifically, these methods and apparatuses may provide cost-optimized medication and pharmacy alternatives for existing prescriptions to prescribers, patients, and/or pharmacists to lower prescription drug costs for payers and patients. The cost-optimized alternative drug and pharmacy information may be created using any of the structured methods and apparatuses described herein. Any of these methods may include the creation and delivery of a structured form referred to herein as a drug savings report (“DSR”).

The structured reporting described herein may be customized (in some cases sorted based on any number of characteristics associated with any prescribed medication) by any of the prescribers, pharmacists, or other authorized users. In some examples, elements of the structured report may be sorted based on a composite rank score. The composite rank score may be a relative score that may be associated with any particular medication. The composite rank score may indicate that the particular medication may have often been substituted in the past with a lower cost, therapeutically equivalent medication.

In some examples, individuals from the patient care team (prescribers, pharmacists, etc.) may collaborate using the DSR to discuss and select alternative medications for the patient based on diagnosis, treatment, and other clinical data. The structured reports may show projected cost savings associated with any particular alternative medication selection.

Any of the methods described herein may be used to generate a drug savings report that includes one or more alternatives for preexisting prescribed medications for a patient. Any of the methods may include receiving a request for to generate the patient's drug saving report, generating a medication list of the patient's preexisting prescribed medications, and identifying a plurality of therapeutically equivalent alternative medications corresponding to one or more of the patient's preexisting prescribed medications, wherein the identification is based on a clinical equivalency of the medications. Any of the methods may further include determining a composite rank score for each preexisting prescribed medication based at least in part on the therapeutically equivalent alternative medications, wherein the composite rank score indicates a historic frequency of substitution of the preexisting prescribed medication with a lower cost therapeutically equivalent medication and providing remote access to users over a network to a drug savings report for a patient, wherein the drug savings report includes preexisting prescribed medications sorted by the composite rank score.

In any of the methods described herein, the composite rank score may be based at least in part on data accessed from a payer claim database, a drug formulary database, a payer rules database, a patient drug cost database, or a combination thereof.

In any of the methods described herein, generating a medication list of the patient's preexisting prescribed medications may be based, at least in part, on a patient's electronic health record.

In any of the methods described herein to generate a drug savings report, identifying a plurality of therapeutically equivalent alternative medications may be based at least in part on prescriber preferences for prescribed medications. In any of the methods, the composite rank score may be further based on the patient's clinical status.

In any of the methods described herein, the drug savings report may be interactively sorted by a user. In some variations, the user may sort the drug savings report based on a cost savings (projected cost savings) per patient. In some other variations, the user may sort the drug savings report based on patient copay costs.

In any of the methods described herein, generating the drug savings report may include determining a cost savings for a medication based on substituting a therapeutically equivalent alternative medication for a patient's preexisting prescription.

The drug savings report may be accessed by pharmacists, prescribers, or other authorized users of a patient's care team. In any of the methods described herein, generating the drug savings report may also include suggesting, by a pharmacist, substitutions for therapeutically equivalent alternative medications for a patient's preexisting prescribed medications based on the drug savings report, calculating a cost savings based on suggested substitutions, and updating the drug savings report based on the calculated cost savings. In some variations, the method may also include transmitting, through the network, the updated drug savings report to the prescriber. In some other variations, calculating the cost savings may include determining an estimated cost savings realized in one year for the suggest substitution. In still other variations, the method may include approving, by a prescriber, the suggested substitutions, and documenting the approved substitutions remotely documented on a server.

In any of the methods described herein, generating the drug savings report may include generating the medication list of the patient's preexisting prescribed medications based on a payer's claim report. In some cases, the method may include determining the composite rank score of a medication during periods of low network activity.

Described herein is a non-transitory computer-readable storage medium storing instructions that, when executed by one or more processors of a device, cause the device to receive a request for to generate a drug saving report, generate a list of a patient's preexisting prescribed medications, identify a plurality of therapeutically equivalent alternative medications corresponding to one or more of the patient's preexisting prescribed medications, wherein the identification is based on a clinical equivalency of the medications, determine a composite rank score for each preexisting prescribed medication based at least in part on the therapeutically equivalent alternative medications, wherein the composite rank score indicates a historic frequency of substitution of a preexisting prescribed medication with a lower cost therapeutically equivalent medication, and provide remote access to users over a network to a drug savings report for a patient, wherein the drug savings report includes preexisting prescribed medications sorted by the composite rank score.

In some examples, a system for generating a drug saving report that includes alternatives for one or more preexisting prescribed medications for a patient may include: a data transceiver, one or more processors; and a memory storing instructions that, when executed by the one or more processors, cause the system to receive a request for to generate a drug saving report, generate a list of a patient's preexisting prescribed medications, identify a plurality of therapeutically equivalent alternative medications corresponding to one or more of the patient's preexisting prescribed medications, wherein the identification is based on a clinical equivalency of the medications, determine a composite rank score for each preexisting prescribed medication based at least in part on the therapeutically equivalent alternative medications, wherein the composite rank score indicates a historic frequency of substitution of a preexisting prescribed medication with a lower cost therapeutically equivalent medication, and provide remote access to users over a network to a drug savings report for a patient, wherein the drug savings report includes preexisting prescribed medications sorted by the composite rank score.

In general, described herein are system and methods for changing a prescribed medication for a patient. As used herein these systems and methods may change the prescribed medication by presenting the patient, caregiver (e.g., prescriber), and/or pharmacist with one or more alternative medications based on a data from a variety of dynamic (e.g., changing) sources, For example, a system may include: one or more processors; and a memory storing instructions that, when executed by the one or more processors, cause the one or more processors to: provide a user interface configured to receive a request for to alternative medications for the patient; identify one or more therapeutically equivalent alternative medications corresponding to one or more of the patient's preexisting prescribed medications based on a clinical equivalency of the alternative and preexisting prescribed medications; build a query batch for two or more of: a current medication database, a clinical content database, a payer formulary database, a payer pharmacy claims database, a payer member eligibility database, and a provider group and NPI database, and independently schedule the two or more queries of the query batch; determine a copay amount, a patient savings and a total savings for each of the one or more therapeutically equivalent alternative medications based on the responses to the two or more queries of the query batch; determine a composite rank score for each preexisting prescribed medication based at least in part on the therapeutically equivalent alternative medications, wherein the composite rank score indicates a historic frequency of substitution of a preexisting prescribed medication with a lower cost therapeutically equivalent medication; and output, in the user interface, a list of a patient's preexisting prescribed medications ranked by the composite rank score.

The instructions may be further configured to cause the one or more processors to request to change the prescribed medication to an alternative medication of the one or more therapeutically equivalent alternative medications. In any of these systems, the instructions may be further configured to cause the one or more processors to output, from an unpublished rebate estimation engine, an unpublished rebate amount for one or more of the therapeutically equivalent alternative medications, wherein the unpublished rebate amount is inferred from historical reply data from prior users, further wherein the total savings includes the unpublished rebate amount for the one or more therapeutically equivalent alternative medications. The unpublished rebate estimation engine may be a part of the instructions (e.g., the software, firmware and/or hardware of the system).

The instructions may be configured to cause the one or more processors to identify the one or more therapeutically equivalent alternative medications using a configurable alternatives engine, as described in greater detail herein.

In some examples, the instructions are configured to cause the one or more processors to build the batch query using a data query and transfer engine. The instructions may be configured to cause the one or more processors to determine the composite rank score based at least in part on one or more of: a payer claim database, a drug formulary database, a payer rules database, a patient drug cost database, or a combination thereof. The instructions may be configured to cause the one or more processors to determine the composite rank score based on the patient's clinical status.

In general, the user interface may present a drug saving report, and may allow the user (e.g., pharmacist) to manipulate the drug saving report, including modifying, sorting or storing (transmitting and/or outputting) the drug savings report. For example, the list of the patient's preexisting prescribed medications may include a drug savings report. In some examples the instructions may be further configured to cause the one or more processors to interactively sort, in response to input from the user interface, the drug savings report.

The instructions may be further configured to cause the one or more processors to independently schedule the two or more queries of the query batch during periods of low network activity for each of the queried databases of the current medication database, clinical content database, payer formulary database, payer pharmacy claims database, and payer member eligibility database.

Also described herein are methods for performing the methods of these systems. For example, a method for changing a prescribed medication for a patient may include: providing a user interface configured to receive a request for to alternative medications for the patient; identifying one or more therapeutically equivalent alternative medications corresponding to one or more of the patient's preexisting prescribed medications based on a clinical equivalency of the alternative and preexisting prescribed medications; building a query batch for two or more of: a current medication database, a clinical content database, a payer formulary database, a payer pharmacy claims database, a payer member eligibility database, and a provider group and NPI database, and independently scheduling the two or more queries of the query batch; determining a copay amount, a patient savings and a total savings for each of the one or more therapeutically equivalent alternative medications based on the responses to the two or more queries of the query batch; determining a composite rank score for each preexisting prescribed medication based at least in part on the therapeutically equivalent alternative medications, wherein the composite rank score indicates a historic frequency of substitution of a preexisting prescribed medication with a lower cost therapeutically equivalent medication; and outputting, in the user interface, a list of a patient's preexisting prescribed medications ranked by the composite rank score.

The method may include requesting to change the prescribed medication to an alternative medication of the one or more therapeutically equivalent alternative medications. Any of these methods may include outputting, from an unpublished rebate estimation engine, an unpublished rebate amount for one or more of the therapeutically equivalent alternative medications, wherein the unpublished rebate amount is inferred from historical reply data from prior users, further wherein the total savings includes the unpublished rebate amount for the one or more therapeutically equivalent alternative medications.

Identifying the one or more therapeutically equivalent alternative medications comprises using a configurable alternatives engine. Building the batch query may include using a data query and transfer engine. Any of these methods may include determining the composite rank score based at least in part on one or more of: a payer claim database, a drug formulary database, a payer rules database, a patient drug cost database, or a combination thereof. In some examples the methods include determining the composite rank score based on the patient's clinical status.

As mentioned, the user interface may present a drug savings report. For example, the list of a patient's preexisting prescribed medications may include or be part of a drug savings report.

Any of these methods may include interactive sorting, in response to input from the user interface, the drug savings report. For example, the method may include independently scheduling the two or more queries of the query batch during periods of low network activity for each of the queried databases of the current medication database, clinical content database, payer formulary database, payer pharmacy claims database, and payer member eligibility database.

As mentioned, it may be particularly helpful to determine and include unpublished rebate estimates in any of these methods and systems. Thus any of these methods and systems may include an unpublished rebate estimation engine that is configured to accurately determine the unpublished rebate estimate. Also described herein are systems for changing a prescribed medication for a patient, the system comprising: one or more processors; and a memory storing instructions that, when executed by the one or more processors, cause the one or more processors to: provide a user interface configured to receive a request for to alternative medications for the patient; identify one or more therapeutically equivalent alternative medications corresponding to one or more of the patient's preexisting prescribed medications based on a clinical equivalency of the alternative and preexisting prescribed medications; output, from an unpublished rebate estimation engine, an unpublished rebate amount for one or more of the therapeutically equivalent alternative medications, wherein the unpublished rebate amount is inferred from historical reply data from prior users; determine a copay amount, a patient savings and a total savings for each of the one or more therapeutically equivalent alternative medications, wherein the total savings includes the unpublished rebate amount for the one or more therapeutically equivalent alternative medications; and output, in the user interface, a list of the patient's preexisting prescribed medications including the copay amount, a patient savings and a total savings.

The system may be configured to cause the one or more processors to sort the list of the patient's preexisting prescribed medications using a composite rank score for each preexisting prescribed medication that is based at least in part on the therapeutically equivalent alternative medications, wherein the composite rank score indicates a historic frequency of substitution of a preexisting prescribed medication with a lower cost therapeutically equivalent medication. In some examples, the instructions are further configured to cause the one or more processors to sort, based on user input from the user interface, the list of the patient's preexisting prescribed medications.

The unpublished rebate estimation engine may be configured to infer the unpublished rebate amount by aggregating estimated approval scores for prior alternative medications for the patient from users of the system. In some examples the unpublished rebate estimation engine may be configured to iteratively update the unpublished rebate amounts for alternative medications based on user feedback. The instructions may be further configured to cause the one or more processors to request to change the prescribed medication to an alternative medication of the one or more therapeutically equivalent alternative medications.

The instructions may be configured to cause the one or more processors to identify the one or more therapeutically equivalent alternative medications using a configurable alternatives engine.

Also described herein are methods for changing a prescribed medication for a patient that include the unpublished rebate amount. For example, a method may include: providing a user interface configured to receive a request for to alternative medications for the patient; identifying one or more therapeutically equivalent alternative medications corresponding to one or more of the patient's preexisting prescribed medications based on a clinical equivalency of the alternative and preexisting prescribed medications; outputting, from an unpublished rebate estimation engine, an unpublished rebate amount for one or more of the therapeutically equivalent alternative medications, wherein the unpublished rebate amount is inferred from historical reply data from prior users; determining a copay amount, a patient savings and a total savings for each of the one or more therapeutically equivalent alternative medications, wherein the total savings includes the unpublished rebate amount for the one or more therapeutically equivalent alternative medications; and outputting, in the user interface, a list of the patient's preexisting prescribed medications including the copay amount, a patient savings and a total savings.

Any of these methods may include sorting the list of the patient's preexisting prescribed medications using a composite rank score for each preexisting prescribed medication that is based at least in part on the therapeutically equivalent alternative medications, wherein the composite rank score indicates a historic frequency of substitution of a preexisting prescribed medication with a lower cost therapeutically equivalent medication. The method may include sorting, based on user input from the user interface, the list of the patient's preexisting prescribed medications.

The unpublished rebate estimation engine may be configured to infer the unpublished rebate amount by aggregating estimated approval scores for prior alternative medications for the patient from users of the system. In some examples the unpublished rebate estimation engine is configured to iteratively update the unpublished rebate amounts for alternative medications based on user feedback.

Any of the methods and systems described herein may include changing he prescribed medication to an alternative medication. Changing the prescribed medication may include requesting to change the prescribed medication to an alternative medication of the one or more therapeutically equivalent alternative medications, which may include providing a DSR.

Also described herein are methods of generating a drug saving report that includes alternatives for one or more preexisting prescribed medications for a patient, the method comprising: receiving a request for to generate the patient's drug saving report; generating a medication list of the patient's preexisting prescribed medications; identifying a plurality of therapeutically equivalent alternative medications corresponding to one or more of the patient's preexisting prescribed medications, wherein the identification is based on a clinical equivalency of the alternative and preexisting prescribed medications; determining a composite rank score for each preexisting prescribed medication based at least in part on the therapeutically equivalent alternative medications, wherein the composite rank score indicates a historic frequency of substitution of the preexisting prescribed medication with a lower cost therapeutically equivalent medication; and providing remote access to users over a network to a drug savings report for a patient, wherein the drug savings report includes preexisting prescribed medications sorted by the composite rank score.

The composite rank score may be based at least in part on data accessed from a payer claim database, a drug formulary database, a payer rules database, a patient drug cost database, or a combination thereof. Generating the medication list of the patient's preexisting prescribed medications may be based, at least in part, on a patient's electronic health record. Identifying a plurality of therapeutically equivalent alternative medications may be based, at least in part, on prescriber preferences for prescribed medications. The composite rank score may be based on the patient's clinical status.

Any of these methods and systems may include interactively sorting, by a user, the drug savings report based at least in part on a cost savings for a patient's medication. For example, any of these methods may include interactively sorting, by a user, the drug savings report by patient copay costs. The methods may include determining a cost savings for a medication based on substituting a therapeutically equivalent alternative medication for a patient's preexisting prescribed medication.

In some examples the methods described herein may include suggesting, by a pharmacist, substitutions for therapeutically equivalent alternative medications for a patient's preexisting prescribed medications based on the drug savings report; calculating a cost savings based on suggested substitutions; and updating the drug savings report based on the calculated cost savings.

Also described herein are software or firmware for performing any of these methods (e.g., non-transitory computer-readable storage medium storing instructions that, when executed by one or more processors of a device, cause the device to perform the method). Also described herein are systems for performing any of these methods; these systems may include one or more processors and the software or firmware for performing the method.

All of the methods and apparatuses described herein, in any combination, are herein contemplated and can be used to achieve the benefits as described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a diagram showing FIG. 1A is a diagram showing an example of a Drug Savings Report (DSR) engine.

FIG. 1B is a diagram showing an example of an automated evaluation request trigger engine.

FIG. 1C is a diagram showing an example of an automated Medications List Collection Engine.

FIG. 1D is a diagram showing an example of an automated Equivalent Alternative Medicine Finder.

FIG. 2 is a diagram showing information used to determine a composite rank score.

FIG. 3 is a flowchart showing an example method for providing or generating a drug savings report DSR.

FIG. 4 is a diagram showing another example of a DSR engine.

FIG. 5 is a diagram showing a relationship between users of a DSR engine.

FIG. 6 is an example screen showing a portion of a DSR.

FIG. 7 shows a block diagram of a device that may be an example apparatus configured to perform operations of the DSR engine of FIG. 1 and/or the DSR engine of FIG. 4.

### DETAILED DESCRIPTION

The methods and apparatuses described herein may determine a number of therapeutically equivalent medications that may be used to replace one or more preexisting prescribed medications that are presently used by a patient. Replacement of the preexisting prescribed medication may provide a cost savings for the patient. In some examples, a drug savings report (DSR) may be generated that lists a patient's prescribed medications and also any associated therapeutically equivalent medications. Along with the equivalent medications, the DSR may present an estimated cost savings that may be realized if a switch is made to the equivalent medication.

Pharmacists and related medical professionals are often tasked with determining reasonable and effective alternatives drugs that have been prescribed to patients, particularly in situations where there may be substantial, and often difficult to identify, differences in pricing and availability for certain drugs. Although there are tools, including computer databases and guides, available to assist the pharmacist in performing this task, these systems are very limited and are not able to track the rapid changes in pricing, discounting, availability, medical and medical professional preferences. The number of variables and the dynamic nature of the problem means that it cannot reasonably be performed by the pharmacist unaided (e.g., mentally). Further, existing systems, including those operating on traditional computer networks and systems, are not able to track changes and identify critical patterns in drug pricing, availability and medical preferences.

The systems and methods described herein include features, including modules and data structures, that are configured specifically to coordinate multiple scheduling and timing parameters so that alternative drugs may be identified, ordered and presented in an interactive user interface for use by the pharmacist. In some examples, these systems and methods may include one or more modules that are configured to aggregate drug pricing data, alternative drug selections, and comments from numerous other users (e.g., pharmacists) using the system, in order to identify hidden (and in some cases, intentionally obscured) drug rebates and incorporate this rebate information into the output of the user interface (e.g., ranking and/or recommending alternative drugs).

For example, the systems described herein may include one or more composite drug rank score engines that generate a score (e.g., rank or aggregate score) that can help the user (e.g., pharmacist) make a recommendation and/or generate the report of alternative drugs. As will be described in greater detail herein, this poses a number of technical problems because the factors that are necessary to generate this score are diverse and change at different time scales, and may not be easily accessible. For example, drug rebates may be unpublished and drug companies often go to great lengths to prevent this information from becoming public. Further, information such as payer formularies, payer preferences, pharmacist preferences, may be maintained in different databases that are updated on different schedules and may be more expensive to access at different times of the day. Other information may be encoded (or encrypted). Other components for drug pricing may include patient clinical status and also medications previously taken (“MPT”) by the patient. This information can be in different databases (some controlled by third parties), that update at different times and are more or less accessible during different parts of the day. As a technical problem it is difficult to access this information from numerous different sources having different updating and cost schedules. The methods and systems described herein include technical solutions, including engines and modules (e.g., pharmacy cost engines, DSR generating engines, medical list collection engines, DSR triggering engines, PBM request modules, configurable alternative engines, incentive/credit calculation engines, and composite rank score engines) that may address these technical problems.

The user interface (pharmacist user interface) described herein also technical solutions to these problems. The dynamic user interfaces described herein are configured so that the pharmacist (user) interactively enters data and sort alternatives using the rank score and one or more other factors (e.g., prescriber, specialty, upcoming dates of appointments, patient savings, total savings, insurance type, etc.) to come up with recommendations. Without the use of this system, one problem is that there are so many options that it is impossible for the pharmacist to know which options are important, and it may be very difficult to navigate this data. Any of these systems may include a sort/rank module (e.g., which may include or be part of the composite rank score determination engine) which may automatically rank order drug alternatives (described in greater detail below). The sort/rank module may be configured to pre-sort the drug alternatives. The user interface may be configured to organize/sort and view/navigate the drug alternatives. The methods and systems described herein may also identify patients/members having the maximum likelihood to reduce their drug costs because they are currently prescribed high scoring/ranked drugs. Finally, these methods and systems may identify prescribers that are most likely to benefit from alternative drugs, e.g., based on the volume of prescribed high scoring/ranked drugs.

Each of the patient's prescribed medications on the DSR may include a composite rank score. The composite rank score may be a relative score that may be associated with any particular medication. In some examples, the composite rank score may indicate that the particular medication may have often been substituted in the past with a lower cost, therapeutically equivalent medication.

Determination of the DSR and/or the composite rank score may be based on data taken from a number of disparate databases, where each database may include vast amounts of patient and/or clinical data. Furthermore, the databases may change frequently making it difficult or impossible for a person to manually remain current with data from so many sources.

In some examples, the DSR may be interactively sorted by a user. The interactive sorting may enable the user to more efficiently manipulate vast amounts of data provided by the DSR to better determine which medication substitutions, if any, provides the best cost savings for the patient.

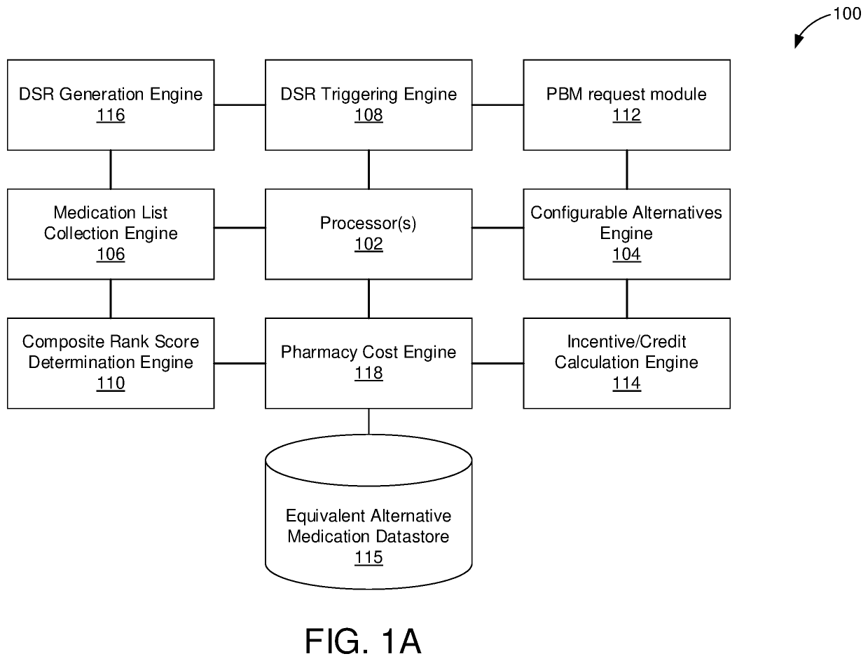

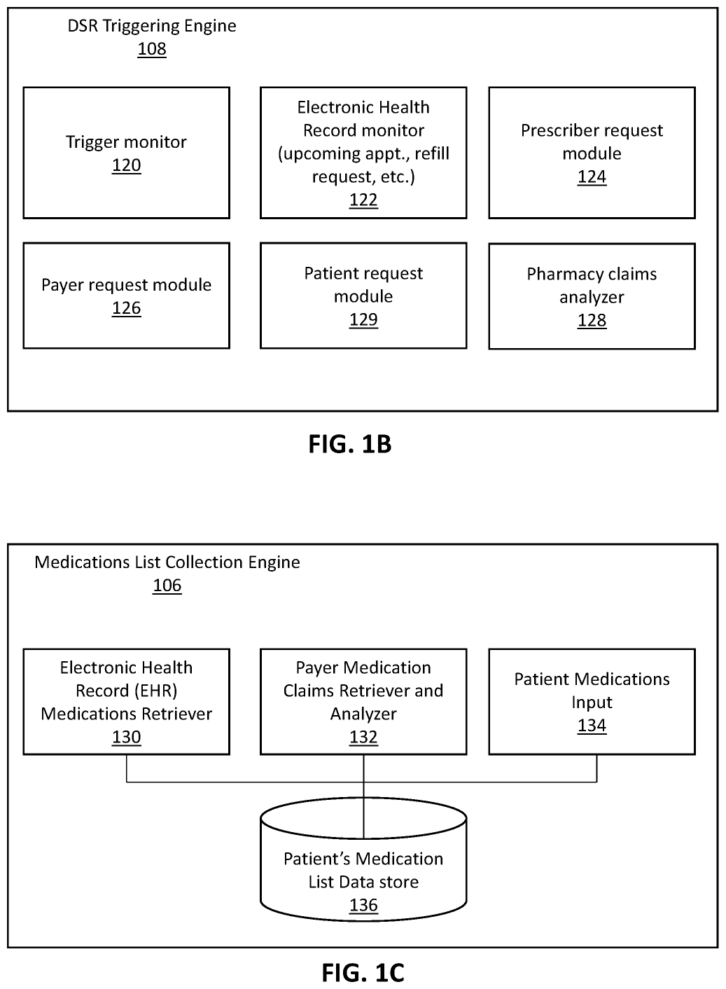

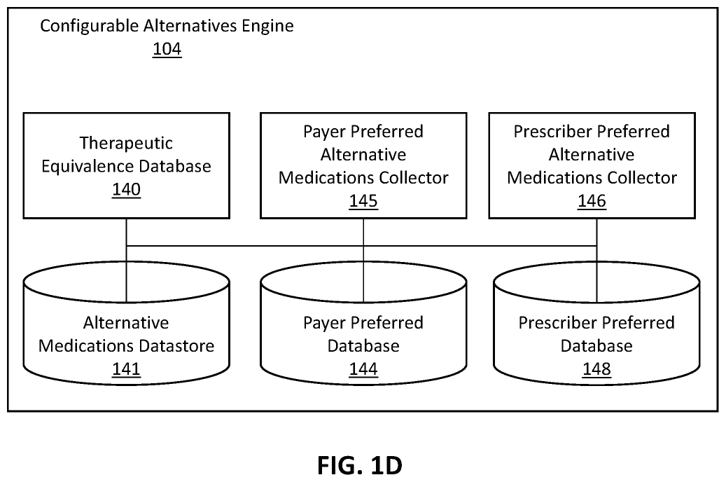

FIG. 1A is a diagram showing an example of a Drug Savings Report (DSR) engine 100. The modules of the DSR engine 100 may include one or more engines and datastores. The DSR engine 100 can be implemented as an engine, as part of an engine, or through multiple engines. As used herein, an engine includes one or more processors 102 or a portion thereof. A portion of the one or more processors 102 can include some portion of hardware, and/or less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized, or its functionality may be distributed. An engine can include hardware, firmware, or software embodied in a computer-readable medium for execution by a processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein can be cloud-based datastores. A cloud-based datastore is a datastore that is compatible with cloud-based computing systems and engines.

The DSR Engine 100 may include a computer-readable medium (not shown) that may be executed on the one or more processors 102, a configurable alternatives engine 104, medication list collection engine 106, a pharmacy benefits manager (PBM) request module 112, an incentive/credit calculation engine 114, a DSR generation engine 116, a DSR triggering engine 108, a composite rank score determination engine 110, a pharmacy cost engine 118, and an equivalent alternative medication datastore 115. The computer-readable medium may include any computer-readable medium, including without limitation a bus, a wired network, a wireless network, or some combination thereof. These modules (engines, datastores, etc.) may be connected to each other in any combination/sub-combination and may be arranged to generate alternatives for one or more preexisting prescribed medications, including generating a report (such as a DSR, not shown).

The equivalent alternative medication datastore 115 may store cross reference information for any feasible medication. For example, for any target medication, the equivalent alternative medication datastore 115 may associate one or more therapeutically equivalent medications that may be substituted for the target medication and provide an equivalent therapeutic effect. The equivalent alternative medication datastore 115 may be maintained by a central authority or by medication providers, or any other feasible provider or payer.

In some variations, data for the alternative medication datastore 115 may be drawn from a number of different databases. Databases may include payer claim databases, formulary databases, provider directories, and patient lists attributed to specific providers. In some cases, the data from other databases may be included within the alternative medication datastore 115 through interactive rules configured by a pharmacist, prescriber, and/or payer that identify, over time, new opportunities for switches from existing medications to lower cost, clinically equivalent medications.

The DSR triggering engine 108 may include one or more modules (e.g., hardware, software, firmware, or a combination thereof) that may cause the DSR generation engine 116 to generate a DSR. The DSR triggering engine 108 is described in more detail with respect to FIG. 1B. The medication list collection engine 106 determines any medications that may be associated with or prescribed to a patient. The medication list collection engine 106 is described in more detail with respect to FIG. 1C.

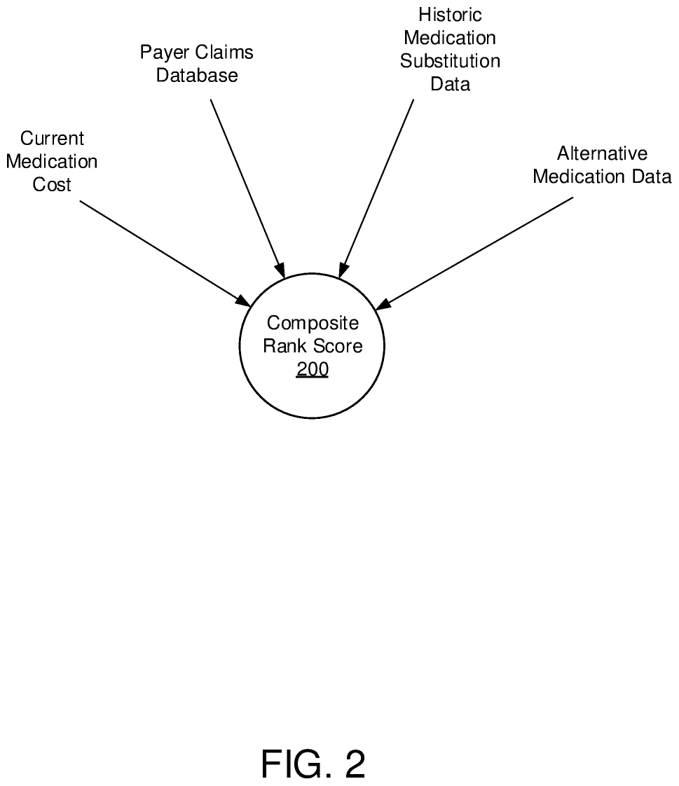

The composite rank score determination engine 110 may generate or determine a relative score that may be associated with any particular medication. In some examples, a high score (or in some cases a low score) may indicate that the particular medication may have often been substituted in the past with a lower cost, therapeutically equivalent medication. For example, some medications may no longer be under patent protection and, therefore, lower cost alternative sources of the medication are available. The composite rank score is described in more detail with respect to FIG. 2.

The pharmacy cost engine 118 may determine patient costs associated with prescribed medications. For example, the pharmacy cost engine 118 may access a patient's electronic health record or other equivalent report that describes the patient's prescribed medications. The pharmacy cost engine 118 may also determine the costs of these medications with respect to various pharmacies available to the patient, and in some cases with regard to out-of-pocket costs for the patient with regard to the patient's health insurance. In some implementations, the pharmacy cost engine 118 can determine the cost of any medication for a patient, even medications not currently prescribed.

The incentive/credit calculation engine 114 determines cost savings and/or cost incentives for any patient. In some examples, the incentive/credit calculation engine 114 can calculate possible savings associated with substituting equivalent alternative medications for some or all of the patient's prescribed medications. In some examples, the incentive/credit calculation engine 114 can access a medication list from the medication list collection engine 106, collect costs associated with the medication list using the equivalent alternative medication datastore 115 and the pharmacy cost engine 118 to determine possible cost savings (sometimes referred to as a shared patient savings (SPS)). The PBM request module 112 can request medication and cost information from a patient's pharmacy benefits manager.

In some embodiments, the DSR engine 100 may determine the patient's pharmacy benefits manager and send customizable data regarding the patient and the prescriber to the patient's pharmacy benefits manager requesting specific information regarding options for lower cost medications. The DSR engine 100 can monitor changes in the patient's medications, lower cost therapeutically equivalent drug alternatives available such as new drugs, drugs going off patent, changes in formularies, changes in rebates, changes in pharmacy coverage and members, or changes in providers who are members of a medical group. The DSR engine 100 enables member or provider-defined time intervals to automatically generate DSRs with predetermined recipients based upon these changes. The DSR engine 100 can further deliver to the pharmacists the exact cost of each relevant alternative drug inclusive of total drug costs, as well as the patient's out-of-pocket costs inclusive of patient's insurance, deductibles, copays and other relevant factors.

In some variations, the DSR engine 100 can not only reduce the cost of prescriptions for patients, payers and provider groups, but also benefit a patient by increasing adherence to medication and preventing delayed onset of care, thereby lowering overall healthcare costs. The systems and methods described herein can further enable a prescriber to focus primarily on patient care, while pharmacist colleagues use pharmaceutical expertise to identify, triage, recommend appropriate lower cost alternative medications, and implement changes to lower cost alternatives once authorized by the prescriber.

The DSR engine 100 can also create additional interactive financial incentives that benefit the patient associated with substituting a lower cost medication by taking a predetermined portion of the payer savings and delivering it to the patient in the form of a credit or copay reduction (e.g., an SPS). The SPS can be dynamic based upon the ratio of patient savings to payer savings or proactively assigned by payers for a given existing medication to an alternative medication switch opportunity. SPS can also be customized based upon a pharmaceutical manufacturer's patient financial assistance program that can reduce or eliminate patient costs and, therefore, patient savings when moving to a lower cost alternative. The redemption of the SPS can be affected either electronically through, for example, the payer's claims processor and/or via a printable SPS coupon or loaded prepaid debit card provided to the patient.

The DSR engine 100 can identify alternative drugs through the configurable alternatives engine 104 that identifies therapeutically equivalent alternative drugs, and then presents the alternatives to a pharmacist or provider based upon payer rules and pharmacist or provider preferences and customizations. Preferences and customizations may be identified by the prescriber themselves, a pharmacist or other intermediary, or automatically through claims data or cost transparency transactions.

FIG. 1B is a diagram showing an example of the DSR triggering engine 108 of FIG. 1A and may include a trigger monitor 120 (monitoring module) that may be configured to receive input from each of the electronic health record monitor 122, a payer request module 126, a prescriber request module 124, and a pharmacy claims analyzer 128. In some examples, the trigger monitor 120 can monitor databases for activity that would trigger an automated outreach to pharmacists or other users suggesting activity. For example, a payer claims database that showed one or more prescriptions for drugs with commonly switched to lower cost alternatives (including high composite rank scores, as described herein) could automatically send an outreach via the DSR engine 100 to a pharmacist suggesting use of the DSR engine 100 for interaction with the prescriber.

The electronic health record (EHR) monitor 112 may monitor a patient's electronic health record (or in some variations a number of different patients/members EHRs) to detect an upcoming appointment with a prescriber, a refill for a prescription request, etc. The EHR monitor 112 may then signal the trigger monitor 120 that such an event has occurred from the EHR, and may pass information including the patient ID/name, date of the appointment/request, and any other additional information. Similarly, the prescriber request module 124 may monitor the prescribers' electronic records to detect a triggering event, such as a medication reconciliation, an appointment for the patient, or a manual request for providing alternatives for one or more preexisting prescribed medications, etc. The payer request module 126 may monitor the payer's electronic records to detect a triggering event, and/or may receive a manual request for providing alternatives for one or more preexisting prescribed medications, a regularly scheduled review and analysis of the patient's preexisting prescribed medicines, etc. In some variations, the DSR triggering engine 108 may also include a patient request module 129 that may retrieve a patient-generated request for an analysis of the patient's current (preexisting) medicines to generate alternatives for one or more preexisting prescribed medications. In addition, the DSR triggering engine 108 may also include a pharmacy claims analyzer 128 that may review pharmacy claims to determine a patient's medication list for use in a drug savings report.

FIG. 1C is a diagram showing an example of the medications list collection engine 106 of FIG. 1A. The medications list collection engine 106 may generate a collection (e.g., list, report, tally, set, etc., which may be sorted or unsorted) of the patient's preexisting prescribed medications. The medication list collection engine 106 may collect this information from the patient's electronic health records via an EHR medication retriever 130. In some variations, the medication list collection engine 106 also may list medications previously taken. Alternatively or additionally, the medication list collection engine 106 may retrieve preexisting prescribed medication information from the patient's paid pharmacy claims via a payer medication claims retriever and analyzer 132. In addition, the patient may themselves report medications via a patient medications input 134, which may provide a user interface for entering medications and may be part of the configurable alternatives engine 104 of FIG. 1A, or may be part of another system, including a prescriber, pharmacy and/or payer system into which the patient enters one or more medications. The patient medications list collection engine 106 may include one or more datastores, including a patient prescribed medications datastore 136, into which prescribed patient medications may be stored, retrieved and/or updated. Note that, in general, the preexisting prescribed medications may include the identity of the medication (e.g., name) as well as patient dosage information (the dose form, the dose concentration, the times/day that the medication is to be taken, etc.).

FIG. 1D is a diagram showing an example of the configurable alternatives engine 104 of FIG. 1A for finding equivalent alternative medicines. In this example, the configurable alternatives engine 104 includes a therapeutic equivalency database 140 as well as input from one or more sources, such as a payer preferred alternative medications collector 145 and/or a prescriber preferred alternative medications collector 146. For example, the payer preferred alternative medications collector 146 may retrieve, update, and/or manage a database of payer preferred alternative medications (e.g., a payer preferred database 144). These alternative medications in this database may be sorted or ranked. The prescriber preferred alternative medications collector 146 may retrieve, update, and/or manage a database of prescriber preferences for alternative medications (e.g., a prescriber preferred database 148). These alternative medications in this database may be sorted or ranked. In some variations, the configurable alternatives engine 104 may use a database to cross-reference the preexisting medications with a listing of alternative medications in the therapeutic equivalency database 140. Alternatives may be prioritized using the payer preferred database 144 and/or prioritized using the prescriber preferred database 148.

In some variations, the configurable alternatives engine 104 may determine and include alternative medicines that should always be included as an alternative medication and determine and exclude alternative medicines that should always be excluded. The inclusion and/or exclusion of medications may be determined from any databases or datastores described herein.

FIG. 2 is a diagram showing information used to determine a composite rank score 200. The composite rank score 200 may be determined by the composite rank score determination engine 110 of FIG. 1A or any other feasible hardware, software, firmware, or combination thereof.

The composite rank score 200 may be a relative score that may be associated with any medication. The score may indicate that a target medication may have one or more therapeutically equivalent substitutions available that may be lower in cost. The score may also indicate that the target medication may have been frequently substituted or replaced with equivalent medications, for example, with respect to other patients. Since the composite rank score 200 may be relative, in some examples a higher score may indicate a lower cost, frequently substituted medication. In some other examples, a lower score may indicate a lower cost, frequently substituted medication. In some implementations, the composite rank score 200 may have a limited or restricted range. For example, the composite rank score 200 may range from 0 to 10. In other examples, the composite rank score 200 may range between any two feasible numbers.

The composite rank score 200 may be based on current medication cost, a payer claims database, historic medication substitution data, and alternative medication data. The current medication cost provides cost information for the target medication as well as current cost information for any therapeutically equivalent medications that may be indicated by the alternative medication data. In this way, the composite rank score 200 may take into account potential cost savings for any particular medication and patient. The payer claims database and historic medication substitution data may provide a history of previously applied or used medication substitutions. In this manner the composite rank score 200 may show or indicate a medication that has been frequently substituted in the past.

In some variations, additional databases may be used alternatively or in conjunction with the payer claims database to determine the composite rank score. For example, the composite rank score 200 may be based on data from a drug formulary database, a payer rules database, and/or a patient drug cost database. In some other variations, the composite rank score may be based on the patient's clinical status. For example, some clinical conditions may be contraindicated with the use for certain alternative medications. The composite rank score may “age” or decrease in relevance based on the passage of time. That is, since the composite rank score may be based on data obtained from numerous databases, and since data in the databases may change frequently, accurate or relevant composite rank scores may need to be current. For example, many databases may be updated frequently (for example, each 24 hours). Therefore, the composite rank score should be calculated and used within the update period (following this example, with 24 hours).

Determination of the composite rank score 200 may involve accessing and processing relatively large amounts of data from disparate databases housed or hosted on disparate computer systems. Thus, the determination of the composite rank score may involve large amounts of data traffic that may increase network (communication) traffic. In some variations, the composite rank score may advantageously be determined during lower network traffic periods (e.g., off-peak times) to avoid network congestion.

In this manner, the composite rank score 200 may be used to indicate to a prescriber or pharmacist that a cost savings may be realized by substituting a different medication for the target medication. In some variations, the magnitude of the composite rank score 200 may be associated with greater potential cost savings and/or more frequently substituted medication.

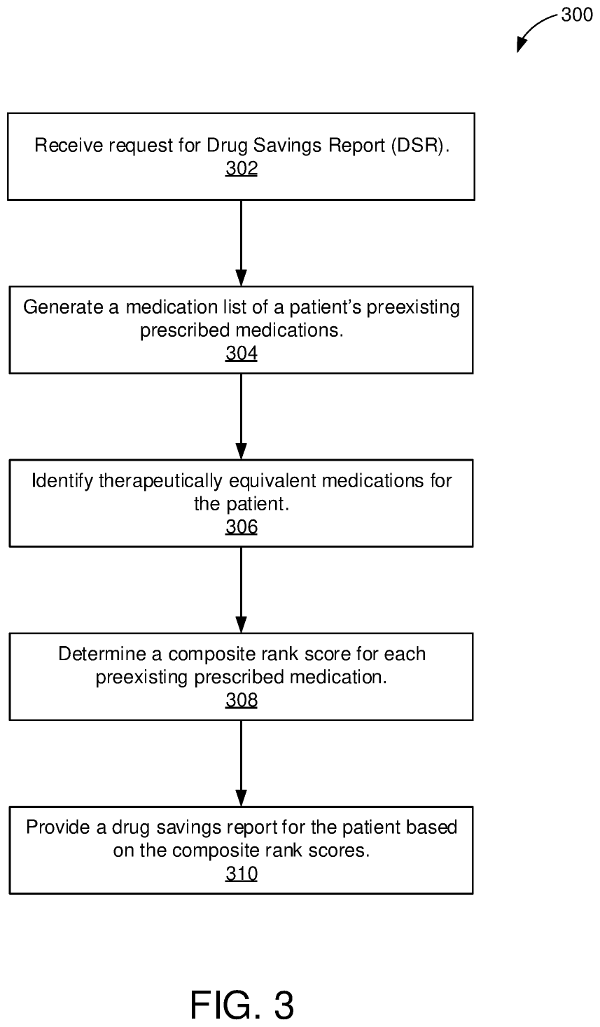

FIG. 3 is a flowchart showing an example method 300 for providing or generating a drug savings report (DSR). Some examples may perform the operations described herein with additional operations, fewer operations, operations in a different order, operations in parallel, and some operations differently. The method 300 is described below with respect to the DSR engine 100 of FIG. 1A, however, the method 300 may be performed by any other suitable system or device.

The method 300 begins in block 302 as the DSR engine 100 receives a request to provide a DSR. For example, the DSR triggering engine 108 may receive an input to generate a DSR from the electronic health record monitor 122, the payer request module 126, the prescriber request module 124, or the pharmacy claims analyzer 128.

Next, in block 304 the DSR engine 100 generates a medication list of a patient's preexisting prescribed medications. For example, the medication list collection engine 106 may retrieve preexisting prescribed medication information from the patient's paid pharmacy claims via the payer medication claims retriever and analyzer 132 (e.g., through a payer's claim report)p. In addition, the patient may themselves report preexisting prescribed medications via the patient medications input 134.

Next, in block 306 the DSR engine 100 identifies therapeutically equivalent medications for the patient. For example, the DSR engine 100 may identify equivalent medications that correspond to one or more of the patient's preexisting prescribed medications. In some implementations, the configurable alternatives engine 104 may provide a number of therapeutically equivalent alternative medications based on the therapeutic equivalency database 140, the payer preferred alternative medications collector 145 and/or the prescriber preferred alternative medications collector 146. In some variations, the configurable alternatives engine 104 may use the payer preferred database 144 and/or the prescriber preferred database 148 to identify therapeutically equivalent alternative medications.

Next, in block 308 the DSR engine 100 determines a composite rank score for each of the patient's preexisting prescribed medications. For example, the composite rank score determination engine 110 may determine a relative score associated with any particular medication that indicates that the particular medication may have often been substituted in the past with a lower cost, therapeutically equivalent medication. In some variations, the composite rank score may be based on data accessed from a payer claim database, a drug formulary database, a payer rules database, or a patient drug cost database.

Next, in block 310 the DSR engine 100 provides a DSR for the patient based on the composite rank scores determined in block 308. In some cases, the DSR may be interactively sorted based on the composite rank scores thereby highlighting medications that may be good candidates for replacement with lower cost, therapeutically equivalent medications.

In some variations, the DSR may be generated on a remotely accessible processor, compute node, computer systems, or the like. For example, the DSR may be generated on a cloud-based computer system accessed remotely through one or more computer networks. Thus, the DSR may be remotely accessible by a pharmacist, a prescriber, and/or a patient.

In some variations, the DSR may be interactively manipulated by a user (prescriber, pharmacist, or the like). For example, the user may sort data on the DSR to present data in a format more useful to the user. In some cases, the user may sort the DSR based on the composite rank score to highlight the medications that are more often substituted with lower cost equivalent medications. In some other cases, the user may sort the DSR based on overall cost savings, or cost savings per medication. In some examples, the user may sort the DSR based on a patient's copay costs.

In some variations, the DSR may be interactively and cooperatively used by multiple users to select substitute medications for a patient. For example, after an initial DSR is generated, the DSR may be reviewed by a pharmacist. Upon review, the pharmacist may suggest one or more medication substitutions for the patient. The suggestions may be provided (transmitted) to the patient's physician (prescriber) through a network. The prescriber may accept (e.g., approve) the suggestions, reject the suggestions, or in some cases, offer different medication substitution suggestions. Any changes to the patient's medication may be documented on a remote device, such as a network-accessible server.

In some examples, new cost savings may be calculated based on the pharmacist's suggestions. The DSR may be updated based on the cost savings. In some variations, the cost savings may be calculated based on savings realized in one year.

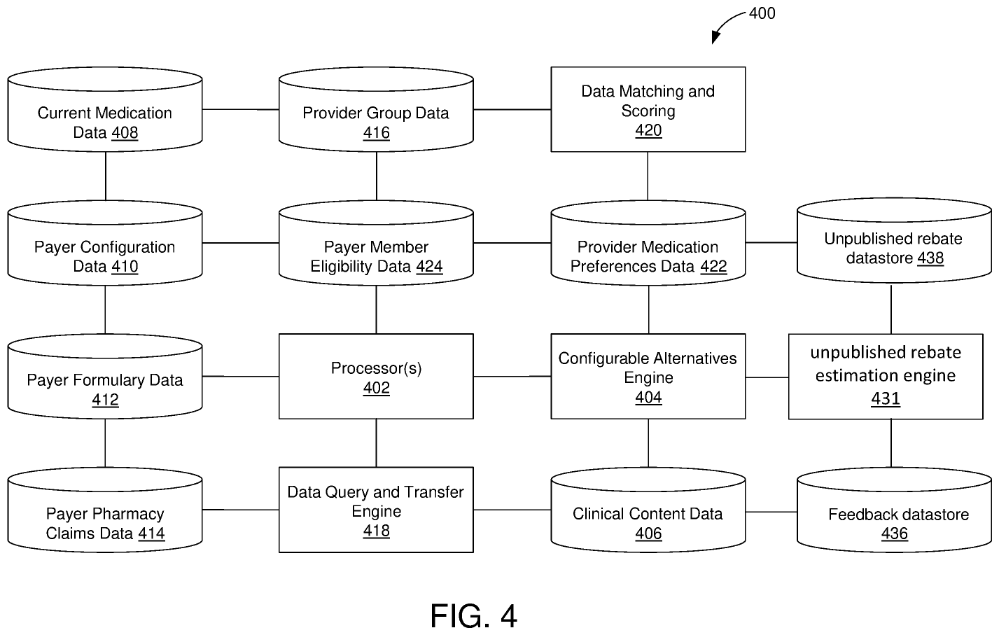

FIG. 4 is a diagram showing another example of a DSR engine 400. The DSR engine 400 may include one or more processors 402, a configurable alternatives engine 404, clinical content data 406, current medication data 408, payer configuration data 410, payer formulary data 412, payer pharmacy claims data 414, provider group data 416, a data query and transfer engine 418, a data matching and scoring module 420, provider medication preferences data 422, and payer member eligibility data 424.

The one or more processors 402 may be similar to the one or more processors 102 of FIG. 1A. Similarly, the configurable alternatives engine 404 may be similar to the configurable alternatives engine 104 of FIG. 1A. In some examples, the configurable alternatives engine 404 may provide or determine the clinical content data 406. Alternatively, or in addition, the configurable alternatives engine 404 may identify a patient's medication as a maintenance or periodic medication, a multi-source or single source medication, and/or identify whether a patient's medication is available in multiple forms which may impact costs. For example, the processor 102 may determine whether a medication is available as a tablet, capsule, cream, ointment, extended release, or the like.

The configurable alternatives engine 404 may identify medications based on a therapeutic category and/or a diagnosis code. In some variations, the configurable alternatives engine 404 identifies conversion factors that enables the determination of dose adjusted alternative medications, as well as associated quantities and number of days supply. In some other variations, the configurable alternatives engine 404 identifies calculates an estimated cost of a chosen (preexisting prescription) medication and each alternative medication. The configurable alternatives engine 404 may determine a National Drug Code associated with the target or the substitution medication.

The current medication data 408 may include information regarding medications currently taken by a patient on an ongoing basis (in some cases as listed in the patient's EHR or as reflected in payer claims). In some examples, the processors 402 may compare claims-based and EHR-based medication lists for reconciliation and/or patient safety purposes.

The payer configuration data 410 may include payer specific rules that determine how the DSR engine 400 interacts with payer formulary data, pharmacy claims data, and configurable alternatives engine data. These rules determine which alternative medications are presented for various scenarios and minimum financial savings thresholds.

The payer formulary data 412 may identify whether a medication is covered or not covered by a patient's health insurance. In some examples, the payer formulary data 412 may indicate the payer's preference for one medication over another medication within a therapeutic category. The payer formulary data 412 may be used by the configurable alternatives engine 404 to align identified alternative medications with the patient's pharmacy benefit formulary by ranking the medications.

In some variations, the payer formulary data 412 is an alternative to ranking alternative medications based on estimated costs and may be used with a payer formulary or default formulary. The payer formulary data 412 may be received from participating pharmacy benefit managers or directly from contracted payers. The payer formulary data 412 may include rebate files to improve estimates of cost savings from alterative medication.

The payer pharmacy claims data 414 may include pharmacy transaction data, both billing and reversal transactions, as delivered to a payer's claims adjudication processing engine by a contracted pharmacy. The payer pharmacy claims data 416 may be used to identify medications filled by the patient, billed to the payer and subsequently dispensed. If a final transaction is a billing transaction, then the medication was billed, dispensed, and acquired by the member. If the final transaction is a reversal transaction, then the medication was returned to stock and not acquired by the member. The payer pharmacy claims data 416 may be used to create and maintain member medication lists and also determine whether an alternative medication was prescribed. In some cases, the member medication lists may identify the patient's current medications. Alternatively, or in addition, the DSR provides a comprehensive list of all medications previously taken by the member for use in considering an alternative medication that may have already been tried.

In some variations, the payer pharmacy claims data 414 may be used to identify the patient's medications previously taken list and can also be used to compare medication lists to the prescriber's EHR medication list. In some other variations, the payer pharmacy claims data 414 may be received from the payer, the payer's pharmacy benefits manager, or pharmacy claims processor agent. The receipt and processing of the payer pharmacy claims data 414 may be automated.

The provider group data 416 may include data regarding medical providers who are members of a specific medical group using DSR services. The medical providers can join a group or leave a group making this database dynamic. The introduction of a new provider would likely trigger the need for dynamic generation of DSRs.

The data query and transfer engine 418 enables any number of databases, datastores, and the like to be queried and transferred between any number of platforms, include high volume data transfer, multiple data formats, and require high utilization of system resources. Additionally, the same data sources may be used by a plurality of unrelated entitles and real time active users for other purposes. In order to optimize system resources and comply with data holder system usage requirements, the data query and transfer engine 418 can batch, queue, and process queries using scheduling logic that optimizes system and data holder resources. Batch schedule processing frees system resources to complete individual real time requests needed on a priority basis.

The data matching and scoring module 420 unifies data resulting from queries into a consolidated patient data file ensuring a dynamic matching logic that identifies, links, and/or organizes these multiple data sources. Further, the sheer quantity of data being communicated may hamper utilization. The data matching and scoring module 420 can utilize scoring logic to prioritize the data based on the highest opportunity for savings utilizing a series of system developed metrics. The resulting data presentation allows for a better utilization of resources toward the greatest opportunity for success.

The provider medication preferences data 422 identifies a provider's medication preferences, if any are applicable. The preferences may be identified and configured manually or automatically through claims data or cost transparency transactions, including alternatives selected by providers and those not selected. The provider medication preferences data 422 may be determined by (i.) passing an indicator to transaction processing services when the chosen medication is a provider's preferred medication, (ii.) passing an indicator to the transaction processing services when an alternative medication is a provider's preferred medication, or (iii.) replacing an identified alternative medication with a provider's preferred medication.

The payer member eligibility data 424 may describe whether any patient is eligible for payments from the payer.

In general, the methods and apparatuses described herein may determine accurate rebate information for one or more drugs even where this rebate information is not explicitly described or is hidden. There are multiple types of drug costs, including out-of-pocket costs and actual costs. In addition, many drugs include rebates, which may be offered particularly with more expensive drugs, and which may be based on the end-of-year (e.g., aggregate) volume of the drug used in the prior year. Thus, many drugs may be divided into rebate tiers, reflecting the fact that the rebate amount may be based on the amount used. Furthermore, these rebates may be independently negotiated by the drug seller (e.g., manufacturer) and the payer or other party, and these negotiated rebates may be confidential (e.g., hidden). Thus, these rebates are unpublished rebates. In practice, such unpublished rebates may be highly significant (e.g., between 5-60% or more). This represents a substantial problem for current systems and methods for estimating alternative drugs, as it may be important to account for such rebates, but since this information is not published (and may be protected behind nondisclosure agreements), it cannot be readily obtained. The methods and systems described herein may provide a technical solution to this problem using an unpublished rebate estimation engine, as shown in FIG. 4.

An unpublished rebate estimation engine may determine an approximate rebate amount by aggregating estimate approval scores from pharmacist using the system. For example, the systems described herein may include an unpublished rebate datastore including estimated rebate amounts for drugs (including alternative drugs). Initially, the estimated rebate amount for the drug(s) may be set at an intermediate value (e.g., 20%, 25%, 30%, etc.) and a starting guess or value. The unpublished estimation engine may automatically modify or adjust this value based on indirect feedback from the users of the system over time. In some examples the estimated rebate amount for the drug(s) may be set within categories (e.g., big, medium, small) and this category may be adjusted by the system based on user feedback for drug saving reports or other user output (e.g., score/ranking output) that includes the particular drug. For example, the user interface (e.g., pharmacist interface) is configured to allow the user to provide input on the quality and/or accuracy of the report and/or rankings. In some examples explicit feedback is not provided, but adoption of alternative recommendations is tracked over time. The unpublished rebate estimation engine 431 may aggregate information from multiple users specific to drugs and may determine an estimate of the accuracy of the current estimated rebate based on this explicit and/or implicit information and may adjust the estimated rebate amount (or range/tier) iteratively over time in order to estimate an accurate estimation. Thus, the estimated drug rebates, which may change over time, may be dynamically and automatically estimated from the overall system data. In some examples the user interface may receive feedback to correct the estimated rebate in this direct or indirect manner and may use this feedback to scale the unpublished estimated rebate for each drug accordingly. The estimated unpublished rebate for any of these drugs may be maintained and adjusted centrally (e.g., from a cloud system, as with any of these systems) and may feed into any of the modules or engines described, including the composite rank score determination engine, DSR generation engine, etc. The user feedback (direct or indirect) may be stored in a datastore (e.g., feedback datastore 436).

In any of these methods and apparatuses, the unpublished rebates may be divided into tiers, as mentioned above. For example, in some examples, the rebate estates may be divided into three tiers that may have associated values; individual drugs may be grouped into one of these tiers. The methods and systems described herein may include a dynamic feedback loop from end users (e.g., pharmacists) which can suggest changes to the one or both of the rebate tier of a class of drugs and/or the amount of the rebate in a tier.

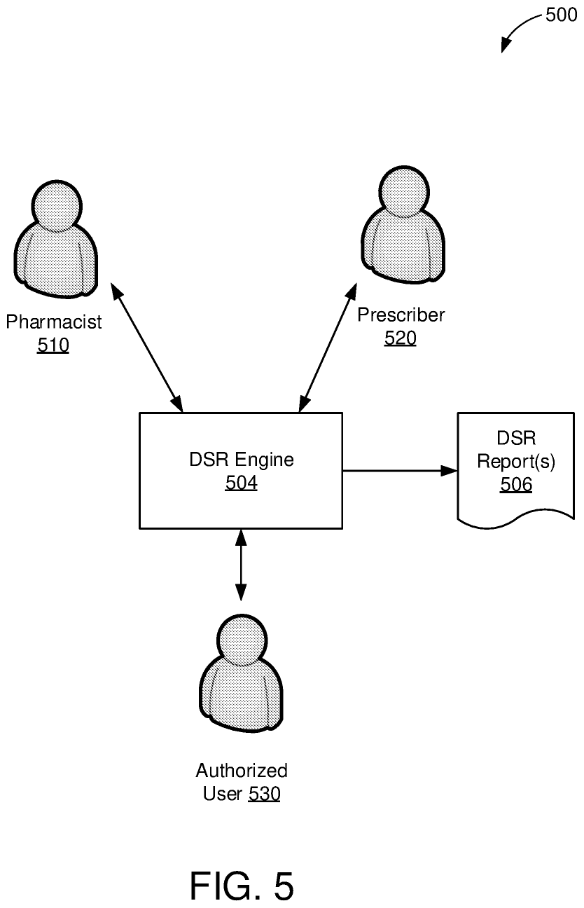

FIG. 5 is a diagram 500 showing the relationship between users of a DSR engine 504. The DSR engine 504 may be an example of the DSR engine 100 of FIG. 1A or the DSR engine 400 of FIG. 4. The DSR engine 504 may generate one or more drug saving reports (DSR) 506 for any number of patients as described herein with respect to FIGS. 1-4. In some examples, two or more users may interactively collaborate to generate, update, and/or revise a DSR 506. For example, a pharmacist 510 may review an initial DSR 506. The pharmacist 510 may sort the DSR 506 based on the composite rank score and make suggestions for medications substitutions. A prescriber 520 may then review the DSR 506 and the suggestions from the pharmacist 510 and may accept or reject the suggestions. In some cases, the pharmacist 510 and the prescriber 520 may go back and forth and iterate changes to the DSR 506. Interactive collaboration may not be limited to the pharmacist 510 and the prescriber 520. For example, any other authorized user 530 may participate in the collaboration.

In some variations, the pharmacist 510 or the prescriber 520 may set a custom reminder to revisit the DSR 506. For example, a custom reminder may include a notification to review the DSR 506 before the end of a 90-day prescription fill period or upon a return of a prescriber 520 from PTO. In some other variations, the DSR engine 504 can include scheduling components that synchronizes to the prescriber's calendar, the electronic health record, and to the pharmacist's calendar.

In some variations, the DSR 506 can be communicated by the pharmacist 510 to the prescriber 520 via fax, email, and/or via the DSR engine 100 and may include integration into video conferencing, or directly into the prescriber's electronic health record. In each case, the DSR 506 can be reviewed and communicated back to the pharmacist 510 with switches to lower cost alternatives or other clinical interventions such as deprescribing or elimination of duplicate therapy authorized by the prescriber 520, thereby allowing the pharmacist 510 to change the prescription, the pharmacy, and notify the patient of the change. The video delivery and discussion of the DSR 506 with the prescriber 520 can also include a recording of the discussion to create a medical-legal record.

In some embodiments, the DSR engine 504 may enable the pharmacist 510 to interactively review lower cost alternatives presented in the DSR 506, customize the alternatives, and then request that the DSR 506 be re-generated with the new pharmacist-selected alternatives and their corresponding costs and savings.

The DSR engine 504 can enable the pharmacist 510 to review custom lower cost alternatives using the DSR 506, including interactivity noting or excluding medications previously taken by the patient based upon the pharmacist 510 or payer selected preferences. The DSR engine 504 can enable the pharmacist 510 to select an alternative diagnosis and then request lower cost alternatives presented in the DSR 506 based upon the selected alternative diagnosis. The pharmacists 510 can focus their work on drugs in the DSR 506 which have a high composite rank score which means that they have a higher chance of being switched by providers with higher total savings per switch.

The DSR engine 504 allows the pharmacist 510 to request a DSR 506 based upon specific prescribers 520, patients, drugs or savings potentials. This can include using the DSR engine 504 and/or accessible databases to identify prescribers with composite rank score, who therefore have higher overall savings opportunities. The pharmacist 510 can then download an interactive file of DSRs 506 and work offline. Alternatively, the pharmacist 510 could review, triage, modify and rerun or mark DSRs as pending via DSR engine 504 without a file download. The pharmacist 510 can also interactively annotate any DSR 506, noting which alternatives they have recommended to the prescribers 520 for switches from current medications. The pharmacist 510 may then upload the DSR 506 via the DSR engine 504 for subsequent confirmation of switches that were made as confirmed by the payer's claims file.

The DSR engine 504 may send planned switches to lower cost alternative medications or other planned activities to be pursued after a discussion between the pharmacist 510 and the prescriber 520 back to the prescriber 520 ahead of a planned discussion noting which switches to lower cost alternative medications (or other medication changes) have been confirmed in related payer claims files or other databases available to the DSR engine 504 and the pharmacist 510.

In some variations, DSR engine 504 allows the pharmacist 510 to identify specific prescribers 520 who are online and automatically send the DSR 506 to the pharmacist 510 for review, edit, comment or recommendations prior to the prescriber 520 seeing the patient and viewing the DSR 506 in their electronic health record. This functionality would allow authorized pharmacists to pre-screen or triage any DSR 506 for patients who are scheduled for care and soon to be seen by prescribers 520.

The DSR engine 504 can forward drug and savings information automatically (in some cases the DSR 506) via an email link, facsimile, or a secure data exchange process, to the prescriber 520 on behalf of the pharmacist 510. These automated messages can be triggered by a pending scheduled discussion between the pharmacist 510 and the prescriber 520 and can include medications and savings opportunities to be discussed.

The DSR engine 504 can automatically send planned medication switches to lower cost alternative medications or other planned activities to be pursued after a discussion between the pharmacist 510 and the prescriber 520 to the prescriber 520 automatically after the discussion. The format for the information sent to the prescriber 520 can be selected by the pharmacist 510 and include the generation of a signable order sheet listing planned medication substitutions or related clinical interventions.

The DSR engine 504 may allow the prescriber 510, the pharmacist 520 and other authorized users 530 to login and see DSRs 506 based upon their, or other users, activity in the DSR engine 504 including switch rates, rates of conversion of recommended switches to actual switches as confirmed in payer claims files, savings for specific time periods or since inception, related professional payments paid or due, comparisons to peers and other reports and information including details on past or upcoming interactions related to the DSR engine 504 and DSR services. These reports can be specific to the user, provider group or payer line of business. The DSR engine 504 can also track deprescribing that occurs. Related activities such as avoidance of prior authorizations can also be tracked through the DSR engine 504.

In some examples, the DSR engine 504 can integrate with third party systems and databases to allow users to see data related to a specific patient or provider. Examples include integration with Medication Therapy Management (MTM) vendors and related clinical applications such as Gaps-in-Care software or databases whereby the user can view information from these third parties within the context of the DSR engine 504.

The DSR engine 504 can export data including one or more DSRs 506 to third party systems and databases on-demand or based upon predetermined rules to allow users of third-party systems to view the DSR 506 and related data specific to a patient or provider within the third-party system. Examples include integration with the MTM vendors and related clinical applications such as Gaps-in-Care software or databases whereby the user can view information DSRs and other data from within these third-party applications and systems.

In some variations, DSR engine 504 allows the pharmacist 510, the prescriber 520 and/or other authorized users 530 to request and receive claims-based medication lists for individual members without inclusion of a complete DSR 506.

The DSR engine 504 allows the pharmacist 510, the prescriber 520, and/or other authorized users 530 to request and receive claims-based medication lists for individual patients, and compare this list to the patients' electronic health record-based list to highlight gaps between the lists to support a medication reconciliation and measure medication adherence and persistence.

The DSR engine 504 can deliver to the pharmacists 510, the prescriber 520, and/or other authorized users 530 aggregate and medication-specific adherence scores that compares the patient's expected medication fills to the actual history. The adherence score can also be an interactive link to the specific medication claims history for one or more medication.

The DSR engine 504 allows administrators to view all activities and reporting through a system dashboard view, including DSRs 506 sent and acted upon that may include resultant savings to the payer. Reporting capabilities include DSRs requested, DSRs delivered, actions taken on each DSR, aggregate actions taken categorized by pharmacists or group of pharmacists, by payer and by prescriber or prescriber groups. Dashboard functionality includes administrator-set automatic notifications, such as a delay in delivering requested DSRs.

The DSR engine 504 can enable the pharmacist 510 and the prescribers 520 to look up a specific patient and request, on demand, DSR information for their existing medications, including mail order and large quantity prescription options.

The DSR engine 504 enables the pharmacist 510 and the prescriber 520 to search for a specific patient and request, on demand, DSR information for a potential new medication, alternate fulfillment options for an existing medication, or alternate quantities.

The DSR engine 504 that allows the pharmacist 510 and prescribers 520 to look up a specific patient and know at a glance if they are on the most cost-effective medications based on their DSR information.

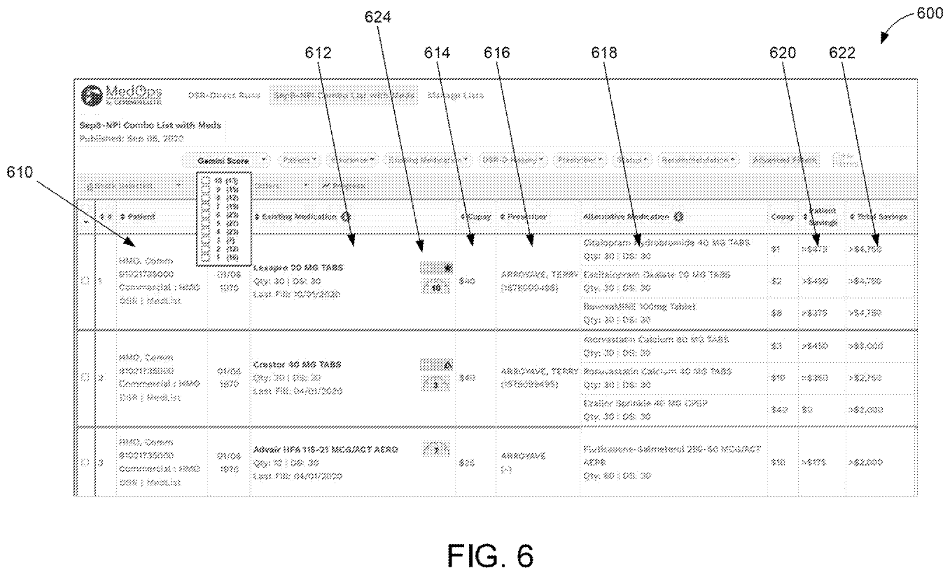

FIG. 6 is an example screen showing a portion of a drug savings report (DSR) 600. The DSR 600 may be generated by any method or engine described herein. The DSR 600 may include information regarding a patient 610, preexisting prescribed medications 612, copay costs 614, prescriber 616, alternative medications 618, projected patient savings 620, and total savings 622. The DSR 600 may include any additional feasible information. For example, the DSR 600 may include a medication last fill date (not shown).

The DSR 600 may be interactively sorted based on any feasible characteristic or included data. For example, the DSR 600 may be sorted by projected patient savings 620 or total (e.g., yearly) savings 622 thereby bringing attention to medication substitutions that may provide the largest cost savings to the patient. In some examples, the DSR 600 may include composite rank scores 624 for each preexisting prescribed medication 612. As described with respect to FIG. 2, composite rank scores 624 can indicate medications that have a history of being substituted to reduce costs. In some examples, the composite rank scores 624 range from 0 up to 10, where larger numbers indicate medications that historically have been substituted with lower cost therapeutically equivalent medications. In some other examples, the composite rank scores 624 range from 10 down to 0, where smaller numbers indicate medications that that historically have been substituted with lower cost therapeutically equivalent medications. In other examples, the composite rank scores 624 may range between any two numbers and lower numbers or higher numbers may indicate medications that have historically been substituted with lower cost therapeutically equivalent medications.

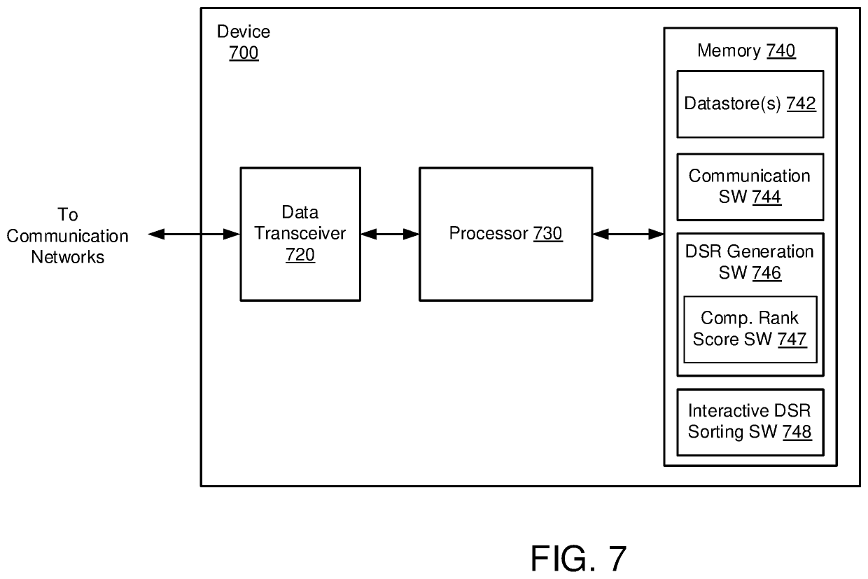

FIG. 7 shows a block diagram of a device 700 that may be an example apparatus configured to perform operations of the DSR engine 100 of FIG. 1 and/or the DSR engine 400 of FIG. 4. The device 700 may include a data transceiver 720, a processor 730, and a memory 740.

The data transceiver 720, which is coupled to the processor 730, may be used to transmit and receive data to and from any feasible device and/or network. In some examples, the data transceiver 720 may include wireline interfaces that transfer data using wire-based communication protocols such as serial, parallel, ethernet protocols, or the like. In some other examples, the data transceiver 720 may include wireless interfaces that transfer data using any feasible wireless protocols such as IEEE 802.11, Bluetooth, ZigBee, cellular communication protocols, or the like.

The processor 730, which is also coupled to the data transceiver 720 and the memory 740, may be any one or more suitable processors capable of executing scripts or instructions of one or more software programs stored in the device 700 (such as within the memory 740).

The memory 740 may include one or more datastores 742. The datastores 742 may include any feasible data (including data and/or datastores described herein) needed to perform operations associated with a DSR engine. For example, the datastores 742 may include an equivalent alternative medication datastore, current medication data, payer configuration data, clinical content data, or the like. In some examples, data for the datastores 742 may be retrieved from other repositories through connected networks and the data transceiver 720.

The memory 740 may also include a non-transitory computer-readable storage medium (e.g., one or more nonvolatile memory elements, such as EPROM, EEPROM, Flash memory, a hard drive, etc.) that may include at least the following software modules: a communication software (SW) module 744, a DSR generation SW module 746, and an interactive DSR sorting SW module 748. Each software module includes program instructions that, when executed by the processor 730, may cause the device 700 to perform the corresponding function(s). Thus, the non-transitory computer-readable storage medium of memory 740 may include instructions for performing all or a portion of the operations described herein.

The processor 730 may execute the communication SW module 744 to communicate with any other feasible devices. For example, execution of the communication SW module 744 may enable the device 700 to communicate via cellular networks, Wi-Fi networks conforming to any of the IEEE 802.11 standards, Bluetooth protocols put forth by the Bluetooth Special Interest Group (SIG), or the like. In some embodiments, execution of the communication SW module 446 may enable the device 700 to communicate via wired networks, and/or with remote (cloud-based) devices through communication networks such as the Internet.

The processor 730 may execute the DSR generation SW module 746 to generate and distribute any feasible DSR. For example, execution of the DSR generation SW module 746 may cause the device 700 to perform operations associated with any DSR engine described herein. Thus, execution of the DSR generation SW module 746 may cause the device 700 to generate and make available a drug savings report for one or more patients. Operations may include execution of any engines described herein to sort and/or process any data, datastore, or database to identify possible therapeutically equivalent medications to replace a preexisting medication used by a patient.

The DSR generation SW module 746 may include a composite rank score SW module 747. Execution of the composite rank score SW module 747 may cause the device 700 to determine a composite rank score for any medication that may be associated with a patient, particularly medications appearing on a DSR provided by the DSR generation SW module 746. The composite rank score may indicate that the medication may have a therapeutically equivalent substitution that may be lower in cost. In some examples, the composite rank score may be the composite rank score 200 of FIG. 2.

The processor 730 may execute the interactive DSR sorting SW module 748 to enable multiple users (e.g., pharmacists, prescribers, other authorized users, etc.) to interactively sort any feasible DSR. For example, execution of the interactive DSR sorting SW module 748 may enable a user to interactively sort any feasible DSR based on any feasible characteristic including, but not limited to, a composite rank score.

### Examples

The systems and methods described herein may include dynamic interactive proposals and/or requests to change drugs, which may be part of a service, including dynamic generation of Drug Savings Reports, that can identify alternative drugs from a configurable alternatives engine that identifies therapeutic equivalent alternative drugs, then presents the alternatives to the pharmacist or provider based upon payer rules and pharmacist or provider preferences, including alternatives that may be presented in response to an existing medication or may never presented either in general or for a specific patient. Preferences may be identified automatically through claims data or cost transparency transactions, include alternatives selected by providers, and those not selected.

Assembling DSR data may involve the transfer of an enormous amount of data across multiple digital platforms. Such high-volume data transfer may tax system resources for both the data holders and the DSR platform. In order to address this challenge, the system may allow for the batching and scheduling of the data transfers to times of lower active system utilization, insuring data transfers are within system resource utilization limitations and are compliant with data holder system usage agreements.

Assembling DSR data may also involve the challenge of matching data from multiple platforms and sources into a unified patient data file. To address this challenge, the system may utilize a dynamic matching logic that is able to identify, link, and organize these multiple data sources into a unified data file which is incorporated into the DSR.

DRS data may also present a challenge to users in the sheer quantity of data being communicated. To transmit the data in the most advantageous format to end users, the system may utilize scoring logic to prioritize the data. The system can score and prioritize data based on the highest opportunity for savings utilizing a series of system developed metrics including drug-specific savings per transaction combined with the historic drug-specific frequency of the transaction (e.g., a composite rank score). The resulting data presentation allows for a better utilization of resources toward the greatest opportunity for success.

The DSRs may provide additional innovation in that they can be interactively configured, requested, viewed, downloaded, sorted, triaged, edited, forwarded to patients or prescribers, placed in a pending file and returned for confirmation of switches by the pharmacist. These interactive features exist within multiple layers of the system, ranging from the original data queries to the preferences of the end user.

Customizable DSR sorting and filtering can include variables including; prescriber, drug, type of savings, date of last fill, amount of patient savings, Gemini Score, and amount of total savings. The interactive DSRs can be customized and edited by the pharmacists by inserting or selecting from a list of preferred alternative drugs which would then change the medication alternatives presented, including recalculating SPS amounts if applicable. Pharmacists can set custom reminders to revisit one or more DSRs. For example, a pharmacist might set a reminder to review a DSR shortly before the end of a 90-day prescription fill period or upon the return of a prescriber from PTO.

The systems and methods for dynamic interactive DSR services, including dynamic generation of Drug Savings Reports, can facilitate transmission of DSRs from pharmacists to prescribers as well as related communication and scheduling of communication. The DSRs can be communicated by the pharmacists to the prescriber via fax, email, via the interactive DSR Platform including integration into video conferencing, or directly into the prescriber's EHR. In each case, the DSR can be reviewed and communicated back to the pharmacist with switches to lower cost alternatives or other clinical interventions such as deprescribing or elimination of duplicate therapy (Switch) authorized by the prescriber; allowing the pharmacists to change the prescription, the pharmacy, and notify the patient of the change. The video delivery and discussion of the DSR with the prescriber can also include a recording of the discussion to create a medical-legal record.

The systems and methods for dynamic interactive DSR services, including dynamic generation of Drug Savings Reports, can include scheduling components that syncs to the prescriber's calendar, the EHR, and to the pharmacist's calendar inside or outside of the DSR Platform. The systems and methods for dynamic interactive DSR services, including dynamic generation of Drug Savings Reports, can not only reduce the cost of prescriptions for patients, payers and provider groups, but also benefit a Patient by increasing adherence to medication and preventing delayed onset of care, thereby lowering overall healthcare costs. The systems and methods for dynamic interactive DSR services can further enable a prescriber to focus primarily on patient care, while their pharmacist colleagues use pharmacy expertise to identify, triage, recommend appropriate lower cost alternative medications, and implement changes to lower cost alternatives once authorized by the prescriber.

The system and method for dynamic interactive DSR services can calculate the annual savings to the Patient and to the payer and/or provider group at risk that would be achieved by substituting a lower cost clinically equivalent alternative medication for an existing medication and presents this information to the pharmacists. The system and method also take the total savings potential represented by all alternative drugs and provides the pharmacist with an estimate of the total potential savings achievable by using all available lower cost alternatives.

The system and method can also create additional interactive financial incentives that benefit the Patient associated with substituting a lower cost medication by taking a predetermined portion of the payer savings and delivering it to the patient in the form of a credit or copay reduction (“Shared Patient Savings” or “SPS”). SPS can be dynamic based upon the ratio of patient savings to payer savings or proactively assigned by payers for a given existing medication to an alternative medication switch opportunity. SPS can also be customized based upon a pharmaceutical manufacturer's patient financial assistance program that can reduce or eliminate patient costs and, therefore, patient savings when moving to a lower cost alternative. The redemption of the SPS can be affected either electronically via the system and method's communication with the payer's claims processor and/or via a printable SPS coupon or loaded prepaid debit card provided to the Patient.

For example, the system and method for dynamic interactive DSR services can use an internal proprietary configurable database and rules engine to identify potential lower cost alternative drugs that the prescriber might consider in treating the Patient. The system and method can then rank and prioritize these alternatives based upon interactive criteria such as payer formularies, payer preferences, pharmacists' preferences, Prescriber preferences. It includes an interactive means of communications related to DSRs, patient clinical status and also medications previously taken (“MPT”) by the patient. This interactive prioritization of alternatives can also include alternatives that should always be presented in response to an existing medication, or never be presented either in general or for a specific patient.

The system and method can determine the Patient's pharmacy benefits manager (“PBM”) and sends customizable data regarding the Patient and the prescriber to the Patient's PBM requesting specific information regarding options for lower cost medications. The system and method can monitor changes in the patient's medications, lower cost therapeutically equivalent drug alternatives available such as new drugs, drugs going off patent, changes in formularies, changes in rebates, changes in pharmacy coverage and members, or changes in providers who are members of a medical group. It allows member or provider-defined time intervals to automatically generate Drug Savings Reports with predetermined recipients based upon these changes. The system and method can further deliver to the pharmacists via an interactive Platform the exact cost of each relevant alternative drug inclusive of total drug costs, as well as the Patient's out-of-pocket costs inclusive of Patient's insurance, deductibles, copays and other relevant factors.

The system and method for dynamic interactive DSR services can allow a pharmacist to interactively review lower cost alternatives presented in the DSR, customize the alternatives, and then request that the DSR be re-generated with the new pharmacist-selected alternatives and their corresponding costs and savings.

The systems and methods for dynamic interactive DSR services, including dynamic generation of Drug Savings Reports, can identify alternative drugs from a Configurable Alternatives engine that identifies therapeutically equivalent alternative drugs, and then presents the alternatives to the pharmacist or provider based upon payer rules and pharmacist or provider preferences and customizations. Preferences and customizations may be identified by the prescriber themselves, a pharmacist or other intermediary, or automatically through claims data or cost transparency transactions.

The system and method for dynamic interactive DSR services can allow a pharmacist to review custom lower cost alternatives using the DSR, including interactivity noting or excluding medications previously taken (“MPT”) by the patient based upon pharmacists or payer selected preferences.

The system and method for dynamic interactive DSR services allows the pharmacists to select an alternative diagnosis and then request lower cost alternatives presented in the DSR based upon the selected alternative diagnosis. Pharmacists can focus their work on drugs in the DSR which have a high Gemini Score which means that they have a higher chance of being switched by providers with higher total savings per Switch.

The system and method for dynamic interactive DSR services allows a pharmacist to request DSR files based upon specific Prescribers, Patients, Drugs or Savings potentials. This can include using the Platform and/or accessible databases to identify prescribers with a high Gemini Score, who therefore have higher overall savings opportunities. A pharmacist can then download an interactive file of DSRs and work offline. Alternatively, a pharmacist could review, triage, modify and rerun or mark DSRs as pending via the Platform without a file download. A pharmacist can also interactively annotate the DSRs, noting which alternatives they have recommended to Prescribers for switches from current medications. A pharmacist may then upload the DSRs via the Platform for subsequent confirmation of switches that were made as confirmed by the payer's claims file.

The system and method for dynamic interactive DSR services allows the pharmacists to communicate DSRs to specific Prescribers via fax, email, via the DSR Platform, in EHRs, or via other interactive means, and with the ability for the prescriber to authorize specific switches to lower cost alternatives and resubmit the DSR to the pharmacist. The pharmacist is then authorized to contact the pharmacy to change the medication to the lower cost alternative and contact the patient confirming the Switch.

The systems and methods for dynamic interactive DSR services, including dynamic generation of Drug Savings Reports, facilitates scheduling of communication between pharmacists and prescribers, including syncing appointments with calendars within the DSR Platform, outside calendars, or schedules within EHRs.

The systems and methods for dynamic interactive DSR services, including dynamic generation of Drug Savings Reports, allows pharmacists to identify specific prescribers who are live on the DSR-E service and have all DSR-Es sent automatically to the pharmacist for review, edit, comment or recommendations prior to the prescriber seeing the patient and viewing the DSR in their EHR. This functionality would allow authorized pharmacists to pre-screen or triage DSRs for patients who are scheduled for care and soon to be seen by prescribers.

The systems and methods for dynamic interactive DSR services, including dynamic generation of Drug Savings Reports, can monitor databases for activity that would trigger automated outreach to pharmacists or other users suggesting activity. For example, a payer claims database that showed one or more prescriptions for drugs with commonly switched to lower cost alternatives (including high Gemini Score drugs) could automatically send an outreach via the Platform to a pharmacist user suggesting use of the Platform for intervention with the prescriber.

The systems and methods for dynamic interactive DSR services, including dynamic generation of Drug Savings Reports, can forward drug and savings information automatically from the Platform, via email link to the Platform, via facsimile, or via a secure data exchange process, to a prescriber on behalf of a pharmacist using the Platform. These automated messages can be triggered by a pending scheduled discussion between a pharmacist user and a prescriber and can include medications and savings opportunities to be discussed.

The systems and methods for dynamic interactive DSR services, including dynamic generation of Drug Savings Reports, can allow the pharmacist user to document in the Platform switches to lower cost alternative medications or other planned activities to be pursued after a discussion between a pharmacist user and a prescriber.

The systems and methods for dynamic interactive DSR services, including dynamic generation of Drug Savings Reports, can automatically send planned switches to lower cost alternative medications or other planned activities to be pursued after a discussion between a pharmacist user and a prescriber to the prescriber automatically after the discussion. The format for the information sent to the prescriber can be selected by the pharmacist user and include the generation of a signable order sheet listing planned switches or related clinical interventions.

The systems and methods for dynamic interactive DSR services, including dynamic generation of Drug Savings Reports, can send planned switches to lower cost alternative medications or other planned activities to be pursued after a discussion between a pharmacist user and a prescriber back to the prescriber ahead of a planned discussion noting which switches to lower cost alternative therapy or other medication changes have been confirmed in related payer claims files or other databases available to the Platform and the pharmacist user.

The systems and methods for dynamic interactive DSR services, including dynamic generation of Drug Savings Reports, can allow prescribers, pharmacists and other authorized users to login and see reports based upon their, or other users, activity in the Platform including Switch rates, rates of conversion of recommended switches to actual switches as confirmed in payer claims files, savings for specific time periods or since inception, related professional payments paid or due, comparisons to peers and other reports and information including details on past or upcoming interactions related to the Platform and DSR services. These reports can be specific to the user, provider group or payer line of business.

The systems and methods for dynamic interactive DSR services, including dynamic generation of Drug Savings Reports, can also track deprescribing that occurs because of Platform and DSR use. Related activities such as avoidance of Prior Authorizations can also be tracked through the Platform.

The systems and methods for dynamic interactive DSR services, including dynamic generation of Drug Savings Reports, can integrate with third party systems and databases to allow users to see data related to a specific patient or provider. Examples include integration with Medication Therapy Management (MTM) vendors and related clinical applications such as Gaps-in-Care software or databases whereby the user can view information from these third parties within the context of the Platform.

The systems and methods for dynamic interactive DSR services, including dynamic generation of Drug Savings Reports, can export data including DSRs to third party systems and databases on-demand or based upon predetermined rules to allow users of third party systems to view DSRs and related Platform data specific to a patient or provider within the third party system. Examples include integration with Medication Therapy Management (MTM) vendors and related clinical applications such as Gaps-in-Care software or databases whereby the user can view information DSRs and other Platform data from within these third parties' applications and systems.

The system and method for dynamic interactive DSR services allows pharmacists and other authorized users to request and receive claims-based medication lists for individual members without inclusion of a full DSR.

The system and method for dynamic interactive DSR services allows pharmacists and other authorized users to request and receive claims-based medication lists for individual members and compare this MedList to the member's EHR-based MedList to highlight gaps between the lists to support medication reconciliation and measure medication adherence and persistence.

The system and method for dynamic interactive DSR services can be delivered to pharmacists and other authorized users aggregate and medication-specific adherence scores that compares the member's expected medication fills to the actual claims history. The adherence score can also be an interactive link to the specific medication claims history for one or more medications.

The system and method for dynamic interactive DSR services may allow administrators to view all activities and reporting in a system Dashboard view, including DSRs sent and acted upon that includes resultant savings to the payer. Reporting capabilities include DSRs requested, DSRs delivered, actions taken on each DSR, aggregate actions taken categorized by pharmacists or group of pharmacists, by Payor and by prescriber or prescriber groups. Dashboard functionality includes administrator-set automatic notifications, such as a delay in delivering requested DSRs. The system and method for a dynamic service that allows pharmacists and prescribers to look up a specific patient and request, on demand, DSR information for their existing medications, including mail order and large quantity prescription options.

The systems and methods for a dynamic service may allow pharmacists and prescribers to search for a specific patient and request, on demand, DSR information for a potential new medication, alternate fulfillment options for an existing medication, or alternate quantities. The system and method for a dynamic service may allow pharmacists and prescribers to look up a specific patient and know at a glance if they are on the most cost-effective medications based on their DSR information.

In any of the systems and method described herein, the system or method may include a dynamic interactive drug savings report services solution that may include all or some of the following ten logical components: Current Medication Data, Clinical Content Data, payer Configuration Data, payer Formulary Data, payer pharmacy Claims Data, payer Member Eligibility Data, Provider Group Data, NPIs, Names, & Practice Types, Data Query and Transfer, Data Matching and Scoring, Provider or Provider Group Medication Preferences Data, Configurable Alternatives Engine software, rules and databases & Interactive Platform.

For example, Current Medication Data may represent medications currently taken by the patient on an ongoing basis as listed in the EHR or as reflected in payer claims. This functionality can include a comparison of claims-based and EHR-based medication lists for reconciliation and/or patient safety purposes. Configurable Alternatives Engine (“CAE”) may be a Prescription alternative identification and management over time used in dynamic generation of Drug Savings Reports data repository used to store clinical content. The CAE may be used to identify clinically appropriate, dose adjusted, alternative medications and corresponding medication Quantities and Days' Supply. Clinical Content Data may include one or more of the following: Identification of whether a medication is recognized as Maintenance or Periodic medication; Identification of whether a medication is recognized as Multi-Source or Single Source, Identification of whether a medication is recognized as a Brand or Generic sourced drug, Identification of whether a medication is available in multiple forms which may impact costs (tablet vs capsule, cream vs ointment, extended release, etc.), Identification of clinically aligned medications via therapeutic category, Identification of clinically aligned medications via diagnosis code (when appropriate), Identification of conversion factors that enables the software to identify dose adjusted Alternative Medications, Quantities and Days' Supply, Identification of cost information that enables the software to calculate an Estimated Cost for the Chosen Medication and each Alternative Medication, and Identification of a Representative NDC, either Brand or Generic, including dosage and form (i.e., tablet vs capsule, cream vs ointment) for each Alternative Medication category.

In addition, in any of these methods and systems, the Configurable Alternatives Engine may be an on-going business function (e.g., updated regularly).

Payer Configuration Data may include the payer specific rules that determine how the system interacts with payer Formulary Data, pharmacy Claims Data, and CAE data. These rules determine which Alternative Medications are presented for various scenarios and minimum financial savings thresholds. Payer Formulary Data may include the Prescription alternative identification and management over time used in dynamic generation of Drug Savings Reports data repository used to store payer formulary content. payer Formulary Data identifies if a medication is covered, or not covered, and the payer's relative preference for one medication over another medication within a therapeutic category. payer Formulary Data is used by the Configurable Alternatives Engine component to align identified Alternative Medications with the Patient's pharmacy benefit formulary by ranking the medications.

Payer Formulary Data may be an alternative to ranking Alternative Medications via Estimated Cost and used with a payer Formulary or Default Formulary. For example, payer Formulary Data may be delivered from participating PBMs, or directly from contracted payers. To the extent possible, the receipt and processing of payer Formulary Data is an automated process. Payer rebate files may also be used to better estimate total savings from alternative drugs.

Payer pharmacy Claims Data may include payer pharmacy Claims Data. Payer pharmacy Claims Data may be the repository that stores pharmacy transaction data, both billing and reversal transactions, delivered to the payer's claims adjudication processing engine by a contracted pharmacy. Payer pharmacy Claims Data may be used to identify medications filled by the patient, billed to the payer and subsequently dispensed. If the final transaction is a billing transaction the medication was billed, dispensed, and acquired by the member. If the final transaction is a reversal transaction the medication was returned to stock and not acquired by the member. Pharmacy Claims Data may be used to create and maintain Member Medication Lists and also determine whether an alternative medication was prescribed. Member Medication Lists identify the patient's current medications. In addition, the DSR may provide a comprehensive list of all medications previously taken by the member for use in considering an alternative medication that may have already been tried.

In addition, Payer pharmacy Claims Data may be used to identify the Patient's MPT, medication list and can be also used to compare medication lists to the prescriber's Electronic Health Record (EHR) medication list. Payer pharmacy Claims Data may be delivered to the provider of the software described herein via the payer or the payer's PBM or pharmacy claims processor agent. The receipt and processing of payer pharmacy Claims Data may be an automated process. Provider Group Data and national provider identifier standard (NPI)-Provider Group data and NPI includes medical providers who are members of a specific medical group using the DSR services. Providers can join a group or leave a group making this database dynamic. The introduction of a new provider would likely trigger the need for dynamic generation of Drug Savings Reports Data Query and Transfer (which may be performed by a Data Query and Transfer engine), may include Current Medication Data, Clinical Content Data, payer Formulary Data, payer pharmacy Claims Data, payer Member Eligibility Data, and Provider group Data and NPIs must be queried and transferred based upon DSR requests. The query and transfer involve multiple platforms, high volume data transfer, multiple data formats, and requires high utilization of system resources. Additionally, the same data sources are utilized by a multitude of unrelated entitles and real time active users for other purposes. In order to optimize system resources and comply with data holder system usage requirements, queries can be batched, queued, and processed utilizing scheduling logic that optimizes system and data holder resources. Batch schedule processing frees system resources to complete individual real time requests needed on a priority basis.

Regarding data matching and scoring, an enormous volume of data is generated through DSR queries. DSR data from multiple platforms and sources can be unified into a consolidated patient data file using a dynamic matching logic that is able to identify, link, and organize these multiple data sources. Further, the sheer quantity of data being communicated hampers utilization. The system can utilize scoring logic to prioritize the data based on the highest opportunity for savings utilizing a series of system developed metrics. The resulting data presentation allows for a better utilization of resources toward the greatest opportunity for success.

The methods and systems may include provider or Provider Group Medication. For example, a provider or provider group (“Providers”) Medication Preferences is the Prescription alternative identification and management over time including dynamic generation of Drug Savings Reports data repository used by the Configurable Alternatives Engine component to identify a Providers medication preferences, if any and applicable. Preferences may be identified and configured manually or automatically through claims data or cost transparency transactions, including alternatives selected by Providers and those not selected. Prescription alternative identification and management over time, including dynamic generation of Drug Savings Reports, supports three or more Providers Medication Preferences scenarios, for example: (i.) Passes an indicator to Transaction Processing Services when the Chosen Medication is a Providers Preferred Medication, (ii.) Passes an indicator to the Transaction Processing Services when an Alternative Medication is a Provider Preferred Medication, or (iii.) Replaces an identified Alternative Medication with a Providers Preferred Medication. The payer may approve the preferred medications. The payer or the provider group may identify the preferred medications.

As discussed above, a Configurable Alternatives Engine (“CAE”) may be part of the Alternative Therapy Messaging component that: receives an Alternative Therapy Request with Chosen Medication, Quantity, and Days' Supply data and pharmacy benefit information (when Patient has pharmacy benefit insurance) and optionally an assigned diagnosis either with the original request or with a subsequent request after an alternative diagnosis has been identified. These may: intersect the Chosen Medication, Quantity, & Days' Supply information and business rules to the Clinical Content data. Intersecting the identified Alternative Medications and the Patient's pharmacy benefit information (i.e., BIN Number, Processor Control Number, Group ID, and Cardholder ID) with the corresponding payer Formulary Data to rank the Alternative Medications, Identifies up to three up Alternative Medications based upon clinical equivalency, payer costs, formularies and rules and provider preferences if any, and may pass a Cost Transparency Response with up to three selected Alternative Medications to the Cost Transparency Processing component.

In some example, the Configurable Alternatives Engine intersects the data and applies the rules that identify the Alternative Medications which may include noting which alternatives are medications previously taken (“MPT”), including the date of last fill. The Configurable Alternatives Engine may intersect with databases that contain drugs available in multiple forms that impact costs such as tablet vs capsule, cream vs ointment, extended release, etc. to optimize the alternative selected.

The Cost Transparency Response data may be used to format and deliver PBM Request transactions to the Patient's pharmacy Benefit Manager or a Drug Savings Card processor. An Interactive Platform may be the Alternative Therapy component that allows pharmacists, Prescribers, Patients or other authorized provider to: register for DSR Platform services, including communication and payment preferences, request DSRs or be notified of DSRs that are ready to be viewed or discussed with pharmacists or other Platform users. This may include: viewing, triage, note as pending, or download DSRs, modify alternatives in DSRs and trigger the DSRs to be regenerate with new alternative drugs, customize alternatives to include pharmacist or provider preferences, including alternatives that should always be presented in response to an existing medication or never presented either in general or for a specific patient, see if a requested DSR has been requested and viewed in the past and, if so, when and by whom with what recommendations resulting, see if a specific drug in a DSR has been changed from a past DSR for the same Patient, may have DSRs in pending files be automatically flagged for the pharmacists based upon pharmacist-selected timing or rules, may have DSRs autogenerated for the pharmacist based upon new Prescribers in their group, new patients under the care of the Prescribers including changes during open enrollment, new medications for patients, changes in payer formularies including rebated drugs; communicate DSRs to Prescribers via fax, email, into the prescriber's EHR in an interactive manner, or other electronic means as specified by the Prescriber, and may receive DSRs once reviewed by the prescriber with switches to lower cost alternatives authorized by the prescriber allowing the pharmacists to change the prescription and the pharmacy and notify the patient of the change. In additional or alternatively, the Prescriber preferences may include previously switched medications changes, never switched medication changes or preferred dates, times and means of communication of recommended alternative switches, and communicate specific lower cost alternatives recommended to Prescribers or Patients for confirmation via the Platform that the recommended Switch was confirmed via payer claims.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for changing a prescribed medication for a patient, the system comprising:
one or more processors; and
a memory storing instructions that, when executed by the one or more processors, cause the one or more processors to:
provide a user interface configured to receive a request for to alternative medications for the patient;
identify one or more therapeutically equivalent alternative medications corresponding to one or more of the patient's preexisting prescribed medications based on a clinical equivalency of the alternative and preexisting prescribed medications;
build a query batch for two or more of: a current medication database, a clinical content database, a payer formulary database, a payer pharmacy claims database, a payer member eligibility database, and a provider group and NPI database, and independently schedule the two or more queries of the query batch;
determine a copay amount, a patient savings and a total savings for each of the one or more therapeutically equivalent alternative medications based on the responses to the two or more queries of the query batch;
determine a composite rank score for each preexisting prescribed medication based at least in part on the therapeutically equivalent alternative medications, wherein the composite rank score indicates a historic frequency of substitution of a preexisting prescribed medication with a lower cost therapeutically equivalent medication; and
output, in the user interface, a list of a patient's preexisting prescribed medications ranked by the composite rank score.

2. The system of claim 1, wherein the instructions are further configured to cause the one or more processors to request to change the prescribed medication to an alternative medication of the one or more therapeutically equivalent alternative medications.

3. The system of claim 1, wherein the instructions are further configured to cause the one or more processors to output, from an unpublished rebate estimation engine, an unpublished rebate amount for one or more of the therapeutically equivalent alternative medications, wherein the unpublished rebate amount is inferred from historical reply data from prior users, further wherein the total savings includes the unpublished rebate amount for the one or more therapeutically equivalent alternative medications.

4. The system of claim 1, wherein the instructions are configured to cause the one or more processors to identify the one or more therapeutically equivalent alternative medications using a configurable alternatives engine.

5. The system of claim 1, wherein the instructions are configured to cause the one or more processors to build the batch query using a data query and transfer engine.

6. The system of claim 1, wherein the instructions are configured to cause the one or more processors to determine the composite rank score based at least in part on one or more of:
a payer claim database, a drug formulary database, a payer rules database, a patient drug cost database, or a combination thereof.

7. The system of claim 1, wherein the instructions are configured to cause the one or more processors to determine the composite rank score based on the patient's clinical status.

8. The system of claim 1, wherein the list of a patient's preexisting prescribed medications comprises a drug savings report.

9. The system of claim 8, wherein the instructions are further configured to cause the one or more processors to interactively sort, in response to input from the user interface, the drug savings report.

10. The system of claim 1, wherein the instructions are further configured to cause the one or more processors to independently schedule the two or more queries of the query batch during periods of low network activity for each of the queried databases of the current medication database, clinical content database, payer formulary database, payer pharmacy claims database, and payer member eligibility database.

11. A method for changing a prescribed medication for a patient, the method comprising:
providing a user interface configured to receive a request for to alternative medications for the patient;
identifying one or more therapeutically equivalent alternative medications corresponding to one or more of the patient's preexisting prescribed medications based on a clinical equivalency of the alternative and preexisting prescribed medications;
building a query batch for two or more of: a current medication database, a clinical content database, a payer formulary database, a payer pharmacy claims database, a payer member eligibility database, and a provider group and NPI database, and independently scheduling the two or more queries of the query batch;
determining a copay amount, a patient savings and a total savings for each of the one or more therapeutically equivalent alternative medications based on the responses to the two or more queries of the query batch;
determining a composite rank score for each preexisting prescribed medication based at least in part on the therapeutically equivalent alternative medications, wherein the composite rank score indicates a historic frequency of substitution of a preexisting prescribed medication with a lower cost therapeutically equivalent medication; and
outputting, in the user interface, a list of a patient's preexisting prescribed medications ranked by the composite rank score.

12. The method of claim 11, further comprising requesting to change the prescribed medication to an alternative medication of the one or more therapeutically equivalent alternative medications.

13. The method of claim 11, further comprising outputting, from an unpublished rebate estimation engine, an unpublished rebate amount for one or more of the therapeutically equivalent alternative medications, wherein the unpublished rebate amount is inferred from historical reply data from prior users, further wherein the total savings includes the unpublished rebate amount for the one or more therapeutically equivalent alternative medications.

14. The method of claim 11, wherein identifying the one or more therapeutically equivalent alternative medications comprises using a configurable alternatives engine.

15. The method of claim 11, further comprising building the batch query using a data query and transfer engine.

16. The method of claim 11, further comprising determining the composite rank score based at least in part on one or more of: a payer claim database, a drug formulary database, a payer rules database, a patient drug cost database, or a combination thereof.

17. The method of claim 11, further comprising determining the composite rank score based on the patient's clinical status.

18. The method of claim 11, wherein the list of a patient's preexisting prescribed medications comprises a drug savings report.

19. The method of claim 18, further comprising interactively sorting, in response to input from the user interface, the drug savings report.

20. The method of claim 18, further comprising independently scheduling the two or more queries of the query batch during periods of low network activity for each of the queried databases of the current medication database, clinical content database, payer formulary database, payer pharmacy claims database, and payer member eligibility database.

21. A system for changing a prescribed medication for a patient, the system comprising:
one or more processors; and
a memory storing instructions that, when executed by the one or more processors, cause the one or more processors to:
provide a user interface configured to receive a request for to alternative medications for the patient;
identify one or more therapeutically equivalent alternative medications corresponding to one or more of the patient's preexisting prescribed medications based on a clinical equivalency of the alternative and preexisting prescribed medications;
output, from an unpublished rebate estimation engine, an unpublished rebate amount for one or more of the therapeutically equivalent alternative medications, wherein the unpublished rebate amount is inferred from historical reply data from prior users;
determine a copay amount, a patient savings and a total savings for each of the one or more therapeutically equivalent alternative medications, wherein the total savings includes the unpublished rebate amount for the one or more therapeutically equivalent alternative medications; and
output, in the user interface, a list of the patient's preexisting prescribed medications including the copay amount, a patient savings and a total savings.

22. The system of claim 21, wherein the instructions are further configured to cause the one or more processors to sort the list of the patient's preexisting prescribed medications using a composite rank score for each preexisting prescribed medication that is based at least in part on the therapeutically equivalent alternative medications, wherein the composite rank score indicates a historic frequency of substitution of a preexisting prescribed medication with a lower cost therapeutically equivalent medication.

23. The system of claim 21, wherein the instructions are further configured to cause the one or more processors to sort, based on user input from the user interface, the list of the patient's preexisting prescribed medications.

24. The system of claim 21, wherein the unpublished rebate estimation engine is configured to infer the unpublished rebate amount by aggregating estimated approval scores for prior alternative medications for the patient from users of the system.

25. The system of claim 21, wherein the unpublished rebate estimation engine is configured to iteratively update the unpublished rebate amounts for alternative medications based on user feedback.

26. The system of claim 21, wherein the instructions are further configured to cause the one or more processors to request to change the prescribed medication to an alternative medication of the one or more therapeutically equivalent alternative medications.

27. The system of claim 21, wherein the instructions are configured to cause the one or more processors to identify the one or more therapeutically equivalent alternative medications using a configurable alternatives engine.

28. The system of claim 21, wherein the list of a patient's preexisting prescribed medications comprises a drug savings report.

29. The system of claim 28, wherein the instructions are further configured to cause the one or more processors to interactively sort, in response to input from the user interface, the drug savings report.

30. A method for changing a prescribed medication for a patient, the method comprising:
providing a user interface configured to receive a request for to alternative medications for the patient;
identifying one or more therapeutically equivalent alternative medications corresponding to one or more of the patient's preexisting prescribed medications based on a clinical equivalency of the alternative and preexisting prescribed medications;
outputting, from an unpublished rebate estimation engine, an unpublished rebate amount for one or more of the therapeutically equivalent alternative medications, wherein the unpublished rebate amount is inferred from historical reply data from prior users;
determining a copay amount, a patient savings and a total savings for each of the one or more therapeutically equivalent alternative medications, wherein the total savings includes the unpublished rebate amount for the one or more therapeutically equivalent alternative medications; and
outputting, in the user interface, a list of the patient's preexisting prescribed medications including the copay amount, a patient savings and a total savings.

31. The method of claim 30, further comprising sorting the list of the patient's preexisting prescribed medications using a composite rank score for each preexisting prescribed medication that is based at least in part on the therapeutically equivalent alternative medications, wherein the composite rank score indicates a historic frequency of substitution of a preexisting prescribed medication with a lower cost therapeutically equivalent medication.

32. The method of claim 30, further comprising sorting, based on user input from the user interface, the list of the patient's preexisting prescribed medications.

33. The method of claim 30, wherein the unpublished rebate estimation engine is configured to infer the unpublished rebate amount by aggregating estimated approval scores for prior alternative medications for the patient from users of the system.

34. The method of claim 30, wherein the unpublished rebate estimation engine is configured to iteratively update the unpublished rebate amounts for alternative medications based on user feedback.

35. The method of claim 30, further comprising requesting to change the prescribed medication to an alternative medication of the one or more therapeutically equivalent alternative medications.

36. The method of claim 30, further comprising identifying the one or more therapeutically equivalent alternative medications using a configurable alternatives engine.

37. The method of claim 30, wherein the list of a patient's preexisting prescribed medications comprises a drug savings report.

38. The method of claim 37, wherein the instructions are further configured to cause the one or more processors to interactively sort, in response to input from the user interface, the drug savings report.
